SEQUENCE LISTING (4 sequences)

SEQ ID NO: 1 - DNA, 336 bp, Artificial Sequence
  Synthetic construct
gatattgtgatgacccagtctccactctctctgtccgtcgcccctggacagccggcctccatctcctgtaagtctagtcagagcctcctggagagttatggagagacctatttgtattggtacctgcagaagccaggccagcctccacagctcctgatctatgcagtttttaagcggttctctggagtgccagataggttcagtggcagcgggtcagggacagatttcacactgaaaatcagccgggtggaggctgaggatgttggggtttattactgcatgcaaagtatgcagcttcctctcactttcggcggagggaccaaggtggagatcaaa

SEQ ID NO: 2 - protein, 112 aa, Artificial Sequence
  Synthetic construct
DIVMTQSPLSLSVAPGQPASISCKSSQSLLESYGETYLYWYLQKPGQPPQLLIYAVFKRFSGVPDRFSGSGSGTDFTLKISRVEAEDVGVYYCMQSMQLPLTFGGGTKVEIK

SEQ ID NO: 3 - DNA, 366 bp, Artificial Sequence
  Synthetic construct
caggtgcagctgcaggagtctgggggaggcgtggtccagcctgggaggtccctgagactctcctgtgcagcctctggattcaccttcaataactttggcatacactgggtccgccaggctccaggcaaggggctggagtgggtggcagttatatcatatgatggaagtgataaatattatgcagactccgtgaagggccgattcaccatctccagagacaattccaagaacacgctgaatctgcaaatgaatagtctgagagctgaggacacggctgtgtattactgtgcgagagatcgtcggtattactaccactactacggtatggacgtctggggccaagggaccatggtcaccgtctcctca

SEQ ID NO: 4 - protein, 122 aa, Artificial Sequence
  Synthetic construct
QVQLQESGGGVVQPGRSLRLSCAASGFTFNNFGIHWVRQAPGKGLEWVAVISYDGSDKYYADSVKGRFTISRDNSKNTLNLQMNSLRAEDTAVYYCARDRRYYYHYYGMDVWGQGTMVTVSS

(19) United States
(12) Patent Application Publication
(10) Pub. No.: US 2024/0156910 A1
(43) Pub. Date: May 16, 2024

(54) TIL ENHANCEMENT VIA EX VIVO STIMULATION WITH CD40 AGONISTS

(71) Applicant: H. LEE MOFFITT CANCER CENTER AND RESEARCH INSTITUTE, INC., Tampa, FL (US)

(72) Inventors: Daniel ABATE DAGA, Tampa, FL (US); Shari PILON-THOMAS, Land O Lakes, FL (US); Renata Marques ROSSETTI, Tampa, FL (US); Matthew BEATTY, Riverview, FL (US)

(21) Appl. No.: 18/282,388

(22) PCT Filed: Mar. 16, 2022
(86) PCT No.: PCT/US2022/020566

§ 371 Date: Sep. 15, 2023

Related U.S. Application Data
(60) Provisional application No. 63/161,753, filed on Mar. 16, 2021.
(60) Provisional application No. 63/253,980, filed on Oct. 8, 2021.

(51) Int. Cl. A61K 38/19; A61K 39/00; C12N 5/0783

(52) U.S. Cl. CPC A61K 38/191; A61K 39/4611; A61K 39/4612; C12N 5/0636; C12N 2501/52

(57) ABSTRACT

In one aspect, disclosed herein are methods of expanding tumor infiltrating lymphocytes (TILs) in vitro or ex vivo comprising obtaining TILs and culturing the TILs in media comprising one or more CD40 agonists (such as, for example, a CD40 ligand (CD40L) and/or anti-CD40 antibody).

This application claims the benefit of U.S. Application No. 63/161,753, filed on Mar. 16, 2021, and U.S. Provisional Application No. 63/253,980, filed on Oct. 8, 2021, applications which are incorporated herein by reference in its entirety.

### I. BACKGROUND

Cluster of differentiation 40 (CD40) mediates many immune activities. Preclinical studies have shown that activation of CD40 can evoke massive antineoplastic effects in several tumor models in vivo, providing a rationale for using CD40 agonists in cancer immunotherapy. To date, several potential agonistic antibodies that target CD40 have been investigated in clinical trials. Early clinical trials have shown that the adverse events associated with agonists of CD40 thus far have been largely transient and clinically controllable, including storms of cytokine release, hepatotoxicity and thromboembolic events. An antitumor effect of targeting CD40 for monotherapy or combination therapy has been observed in some tumors. However, these antitumor effects have been moderate. There is therefore a need for biomarkers for monitoring and predicting responses and informing resistance mechanisms.

### II. SUMMARY

In one aspect, disclosed herein are methods of expanding tumor infiltrating lymphocytes (TILs) in vitro ex vivo comprising obtaining TILs and culturing the TILs in media comprising one or more CD40 agonists (such as, for example, a CD40 ligand (CD40L) and/or anti-CD40 antibody). In one aspect, the TILs can be cultured for at least 21 days.

Also disclosed herein are methods of expanding TILs of any preceding aspect, further adding autologous B cells to the culture of TILs and CD40L.

In one aspect, disclosed herein are methods of expanding tumor infiltrating lymphocytes (TILs) in in a subject with a tumor comprising administering one or more CD40 agonists (such as, for example, a CD40 ligand (CD40L) and/or anti-CD40 antibody) to the subject at the site of the tumor.

In one aspect, disclosed herein are methods of treating, inhibiting, reducing, decreasing, ameliorating, and/or preventing a cancer and/or metastasis in a subject comprising obtaining tumor infiltrating lymphocytes (TILs) from the subject; culturing the TILs in media comprising one or more CD40 agonists, and administering the cultured TILs to the subject. In one aspect, the TILs can be cultured for at least 21 days.

Also disclosed herein are methods of treating, inhibiting, reducing, decreasing, ameliorating, and/or preventing a cancer and/or metastasis of any preceding aspect, further comprising measuring the tumor gene expression level of chemokine (C-C motif) ligand 2 (CCL2), CCL3, CCL4, CCL5, CCL8, chemokine (C-C motif) ligand 18 (pulmonary and activation-regulated) (CCL18), CCL19, CCL21, chemokine (C-X-C motif) ligand 9 (CXCL9), CXCL10, CXCL11, and CXCL13 in tumor cells and comparing the tumor gene expression levels to reference gene expression levels; and identifying a subject who has tumor gene expression levels above the reference gene expression levels.

In one aspect, also disclosed herein are methods of treating, inhibiting, reducing, decreasing, ameliorating, and/or preventing a cancer and/or metastasis of any preceding aspect, further comprising adding autologous B cells to the culture of TILs and the one or more CD40 agonists.

### III. BRIEF DESCRIPTION OF THE DRAWINGS

The accompanying drawings, which are incorporated in and constitute a part of this specification, illustrate several embodiments and together with the description illustrate the disclosed compositions and methods.

FIG. 1 shows TIL enhancement via ex vivo stimulation with CD40 agonists

FIGS. 2A and 2B show TIL expansion from melanoma digests is improved by CD40 stimulation. Frozen tumor digests were cultured in presence of high-dose IL-2 (control) or high-dose IL-2 plus a CD40 agonist (for 4 weeks. The total number of live cells (2A), CD4+T lymphocytes (2B), and of CD8+ lymphocytes was significantly increased in the CD40 stimulated group. *p<0.05.

FIGS. 3A and 3B show melanoma tumors contain B cells, which can be activated using a CD40 agonist. FIG. 3A shows frozen tumor digests were analyzed for the presence of B cells (CD19+) and myeloid cells (HLA-DR^{hi}). The vast majority of B cells expressed CD40, while half of myeloid cells did. FIG. 3B shows tumor digests were cultured for two days in presence of standard TIL expansion media (Ctrl.) or the same media supplemented with a CD40 agonist (CD40 stim). Flow cytometry analysis showed induction of CD80 and CD86 costimulatory ligands by the CD40 agonist. HLA molecules were detected in all B cells in resting and stimulated conditions, but the intensity of HLA-DR expression was increased in the CD40-stimulated samples.

FIG. 4 shows TIL expansion from melanoma digests is improved by CD40 stimulation. Frozen tumor digests were cultured in presence of high-dose IL-2 (control) or high-dose IL-2 plus a CD40 agonist (for 4 weeks. The total number of live cells, CD4+T lymphocytes, and of CD8+ lymphocytes was significantly increased in the CD40 stimulated group. *p<0.05.

FIGS. 5A and 5B show that CD40 stimulation results in increased Effector Memory cells, and increased CD39-cells in 3/5 cases. Frozen tumor digests were cultured in presence of high-dose IL-2 (control) or high-dose IL-2 plus a CD40 agonist (for 4 weeks. FIG. 5A shows analysis of differentiation phenotype based on CD45RA and CCR7 expression. FIG. 5B shows expression of CD39 and CD69 in the CD4 T cell compartment. *p<0.05.

FIG. 6 shows TIL growth. The number of NSCLC samples in which TIL expanded vs not expanded. Patient Samples from NSCLC were dissociated, and TIL expanded in conditions containing IL-2 or IL-2+CD40L.

FIG. 7 shows the expansion of TIL and CD4 CD8 phenotype. Patient Samples from NSCLC were dissociated, and TIL expanded in conditions containing IL-2 or IL-2+CD40L. Absolute number of TIL expanded was compared as well as CD4 and CD8 percentages as analyzed by flow cytometry.

FIG. 8 shows the stem like and exhausted phenotype. TIL expanded from NSCLC samples with IL-2 or IL-2+CD40L were analyzed for (A) stem like markers (CD39− CD69−) and (B) exhausted markers (CD39+CD69+) by flow cytometry.

FIG. 9 shows TIL Reactivity. TIL from NSCLC samples expanded in IL-2 or IL-2+CD40L were co-cultured with dissociated autologous tumor cells at a ratio of 1:1. An MHC-I blocking antibody was used for analysis of MHC-I specificity of reaction. After 24 hrs post culture, supernatant was collected and analyzed for Granzyme B, IFNg, and TNFa expression by use of the Protein Simple ELLA platform.

### IV. DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. DEFINITIONS

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

“Optional” or “optionally” means that the subsequently described event or circumstance may or may not occur, and that the description includes instances where said event or circumstance occurs and instances where it does not.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

“Treat,” “treating,” “treatment,” and grammatical variations thereof as used herein, include the administration of a composition with the intent or purpose of partially or completely preventing, delaying, curing, healing, alleviating, relieving, altering, remedying, ameliorating, improving, stabilizing, mitigating, and/or reducing the intensity or frequency of one or more a diseases or conditions, a symptom of a disease or condition, or an underlying cause of a disease or condition. Treatments according to the invention may be applied preventively, prophylactically, pallatively or remedially. Prophylactic treatments are administered to a subject prior to onset (e.g., before obvious signs of cancer), during early onset (e.g., upon initial signs and symptoms of cancer), or after an established development of cancer. Prophylactic administration can occur for day(s) to years prior to the manifestation of symptoms of an infection.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

“Biocompatible” generally refers to a material and any metabolites or degradation products thereof that are generally non-toxic to the recipient and do not cause significant adverse effects to the subject.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

A “control” is an alternative subject or sample used in an experiment for comparison purposes. A control can be “positive” or “negative.”

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

“Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

A “pharmaceutically acceptable” component can refer to a component that is not biologically or otherwise undesirable, i.e., the component may be incorporated into a pharmaceutical formulation provided by the disclosure and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition (e.g., a non-immunogenic cancer). The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

“Therapeutically effective amount” or “therapeutically effective dose” of a composition (e.g. a composition comprising an agent) refers to an amount that is effective to achieve a desired therapeutic result. In some embodiments, a desired therapeutic result is the control of type I diabetes. In some embodiments, a desired therapeutic result is the control of obesity. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as pain relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. METHODS OF EXPANDING TILS AND USING EXPANDED TILS FOR THE TREATMENT OF CANCER

Tumor infiltrating lymphocytes (TILs) are mononuclear cells that have left the bloodstream and migrated into a tumor. TILs have been used in autologous adaptive transfer therapy for the treatment of cancer. Typically, a fresh surgically resected tumor is used as the starting material for successful initiation and expansion of tumor specific TIL culture to manufacture a clinically relevant dose of TIL therapy. Therefore, the candidate patient for TIL therapy needs to be eligible for surgery. If the patient is eligible for surgery, the tumor needs to be resectable. If several tumor anatomical sites are present, a skilled choice of resection of the suitable tumor sites with potential T cell infiltration must be made for each patient.

In the production of TILs, once a surgically resectable tumor has been obtained, 5-7 weeks of culture are needed and the culture conditions necessitate the use of a cleanroom, splitting of cultures to check confluence, and considerable time to maintain the cells. Disclosed herein are more rapid methods of expanding TILs.

In one aspect, disclosed herein are methods of expanding tumor infiltrating lymphocytes (TILs) in vitro or ex vivo comprising obtaining TILs and culturing the TILs in media comprising one or more CD40 agonists (such as, for example, a CD40 ligand (CD40L) and/or anti-CD40 antibody). In one aspect, the TILs can be cultured for at least 3, 4, 5, 6, 7, 8, 9, 10, 11, 12, 13, 14, 15, 16, 17, 18, 19, 20, 21, 22, 23, 24, 25, 26, 27, 28, 29, 30, or 31 days.

It is understood and herein contemplated that the TILs are obtained from a biopsy (such as, for example, core biopsies and/or one or more surgical resections) of a surgically resectable tumor. In one aspect, the disclosed methods of producing an expanded TIL population comprise obtaining one or more biopsies from the subject (such as, for example, percutaneous tumor samples). As used herein, “biopsy” can include any partial removal of a tissue such as excisional, incisional, core, or fine needle aspiration biopsies. It is understood and herein contemplated that the use of TILs obtained from biopsies (such as, for example, core biopsies including core needle biopsies) makes TIL therapy available to patients who are not eligible for surgery and for patients with unresectable tumors. In addition, core biopsies (such as, for example core needle biopsies) allows for image guided sampling from several anatomical sites.

Where biopsies, and in particular, core biopsies are used as the source of the tissue sample, it is understood and herein contemplated that core biopsies (such as, for example core needle biopsies) can be obtained using any device with which a core biopsy can be obtained (see, for example, the Bard Core Biopsy Instruments and Temno Biopsy Systems by Carefusion such as, BARD MAGNUM®, BARD MAX-CORE®, BARD BIOPTY-CUT®, BARD MARQUEE®, BARD MISSION®, and BARD MONOPTY® from CR Bard, Inc.). The needle for obtaining the biopsy can be 6, 8, 10, 12, 14, 16, 18, or 20 gauge needle with a needle length between about 2 cm and to about 30 cm long, preferably between about 10 cm and about 25 cm long, more preferably between about 16 cm and about 20 cm long. For example, the needle length for obtaining a core biopsy can be 2, 3, 4, 5, 6, 7, 8, 9, 10, 11, 12, 13, 14, 15, 16, 17, 18, 19, 20, 21, 22, 23, 24, 25, 26, 27, 28, 29, or 30 cm long. The penetration depth of the needle can be between about 15 mm and 30 mm, preferably between about 20 mm and 25 mm. For example, the penetration depth can be 15, 16, 17, 18, 19, 20, 21, 22, 23, 24, 25, 26, 27, 28, 29, or 30 mm.

In one aspect, the use of core biopsy allows the ability to target certain and possibly multiple areas of a tumor. In one aspect, disclosed herein are methods of rapidly producing an expanded TIL population further comprising the use of imaging techniques such as radiomics to guide TIL acquisition.

Once obtained, tissue samples, including, but not limited to biopsies (such as, for example, core biopsies including core needle biopsies) and/or surgical resections provide the added advantage of not requiring further sectioning (i.e., making fragments), but can be directly digested. In one aspect, the disclosed methods can comprise placing the tissue sample directly into a digesting solution (such as, for example a at least one, or a combination of two, or all three of the human or humanized enzymes selected from the group consisting of collagenase (e.g., XIAFLEX®), hyaluronidase (e.g., HYLENEX®), and DNAse (e.g., PULMOZYME®)).

The tumor cells can be dissociated using mechanical fragmentation (either pooled fragments or separated) and/or using a tumor digest. Accordingly, disclosed herein are methods of expanding tumor infiltrating lymphocytes (TILs), wherein the one or more core biopsies are digested directly from the patient without disaggregation of the specimen. Digests are well known in the art and typically comprise a collagenase, hyaluronidase, and DNAse. It is understood and herein contemplated that the digest can comprise at least one, or a combination of two, or all three of the human or humanized enzymes selected from the group consisting of collagenase (e.g., XIAFLEX®), hyaluronidase (e.g., HYLENEX®), and DNAse (e.g., PULMOZYME®). As noted above, in some instances the digest can occur directly from the tissue sample without prior fragmentation (mechanical or otherwise) or the separation or pooling of fragments thereby creating a bulk non-purified digest.

The concentration of fragmented and/or digested cells used in the pre-REP of the disclosed methods can affect the yield and or efficacy of the disclosed methods. In one aspect, the methods utilizes less than 5×10⁶ cells, for example, the method can use 4×10⁶, 3×10⁶, 2×10⁶, 1×10⁶, 9×10⁵, 8×10⁵, 7×10⁵, 6×10⁵, 5×10⁵, 4×10⁵, 3×10⁵, 2×10⁵, or 1×10⁵ or less bulk non-purified digest cells per tissue culture well.

Also disclosed herein are methods of expanding TILs, further adding autologous B cells to the culture of TILs and CD40L.

In one aspect, disclosed herein are methods of expanding tumor infiltrating lymphocytes (TILs) in in a subject with a tumor comprising administering one or more CD40 agonists (such as, for example, a CD40 ligand (CD40L) and/or anti-CD40 antibody) to the subject at the site of the tumor.

By “CD40 antigen” is intended a glycosylated transmembrane peptide or any fragment thereof (GenBank Accession No. X60592; U.S. Pat. Nos. 5,674,492 and 4,708,871; Stamenkovic et al. (1989) EMBO 8:1403; Clark (1990) Tissue Antigens 36:33; Barclay et al. (1997) The Leucocyte Antigen Facts Book (2d ed.; Academic Press, San Diego)). The CD40 receptor is displayed on the surface of a variety of cell types, as described elsewhere herein. By “displayed on the surface” and “expressed on the surface” is intended that all or a portion of the CD40 antigen is exposed to the exterior of the cell. The displayed or expressed CD40 antigen may be fully or partially glycosylated.

By “agonist activity” is intended that the substance functions as an agonist. An agonist combines with a receptor on a cell and initiates a reaction or activity that is similar to or the same as that initiated by the receptor's natural ligand. An agonist of CD40 induces any or all of, but not limited to, the following responses: B-cell proliferation and differentiation, antibody production, intercellular adhesion, B-cell memory generation, isotype switching, up-regulation of cell-surface expression of MHC Class II and CD80/86, and secretion of pro-inflammatory cytokines such as IL-8, IL-12, and TNF.

By “antagonist activity” is intended that the substance functions as an antagonist. An antagonist of CD40 prevents or reduces induction of any of the responses induced by binding of the CD40 receptor to an agonist ligand, particularly CD40L. The antagonist may reduce induction of any one or more of the responses to agonist binding by 5%, 10%, 15%, 20%, 25%, 30%, 35%, preferably 40%, 45%, 50%, 55%, 60%, more preferably 70%, 80%, 85%, and most preferably 90%, 95%, 99%, or 100%. Methods for measuring B-cell responses are known to one of skill in the art and include, but are not limited to, B-cell proliferation assays, Banchereau-Like-B-Cell proliferation assays, T-cell helper assays for antibody production, co-stimulation of B-cell proliferation assays, and assays for up-regulation of B-cell activation markers. Several of these assays are discussed in more detail elsewhere herein.

By “significant” agonist activity is intended an agonist activity of at least 30%, 35%, 40%, 45%, 50%, 60%, 70%, 75%, 80%, 85%, 90%, 95%, or 100% greater than the agonist activity induced by a neutral substance or negative control as measured in an assay of a B-cell response. A substance “free of significant agonist activity” would exhibit an agonist activity of not more than about 25% greater than the agonist activity induced by a neutral substance or negative control, preferably not more than about 20% greater, 15% greater, 10% greater, 5% greater, 1% greater, 0.5% greater, or even not more than about 0.1% greater than the agonist activity induced by a neutral substance or negative control as measured in an assay of a B-cell response. The antagonist anti-CD40 antibodies useful in the methods of the present invention are free of significant agonist activity as noted above when bound to a CD40 antigen on a normal human B cell. In one embodiment of the invention, the antagonist anti-CD40 antibody is free of significant agonist activity in one B-cell response. In another embodiment of the invention, the antagonist anti-CD40 antibody is free of significant agonist activity in assays of more than one B-cell response (e.g., proliferation and differentiation, or proliferation, differentiation, and antibody production).

As used herein “anti-CD40 antibody” encompasses any antibody that specifically recognizes the CD40 B-cell surface antigen, including polyclonal antibodies, monoclonal antibodies, single-chain antibodies, and fragments thereof such as Fab, F(ab′)2, Fv, and other fragments which retain the antigen binding function of the parent anti-CD40 antibody. Polyclonal sera may be prepared by conventional methods. In general, a solution containing the CD40 antigen is first used to immunize a suitable animal, preferably a mouse, rat, rabbit, or goat. Rabbits or goats are preferred for the preparation of polyclonal sera due to the volume of serum obtainable, and the availability of labeled anti-rabbit and anti-goat antibodies. Polyclonal sera can be prepared in a transgenic animal, preferably a mouse bearing human immunoglobulin loci. In a preferred embodiment, Sf9 cells expressing CD40 are used as the immunogen. Immunization can also be performed by mixing or emulsifying the antigen-containing solution in saline, preferably in an adjuvant such as Freund's complete adjuvant, and injecting the mixture or emulsion parenterally (generally subcutaneously or intramuscularly). A dose of 50-200 μg/injection is typically sufficient. Immunization is generally boosted 2-6 weeks later with one or more injections of the protein in saline, preferably using Freund's incomplete adjuvant. One may alternatively generate antibodies by in vitro immunization using methods known in the art, which for the purposes of this invention is considered equivalent to in vivo immunization. Polyclonal antisera are obtained by bleeding the immunized animal into a glass or plastic container, incubating the blood at 25° C. for one hour, followed by incubating at 4° C. for 2-18 hours. The serum is recovered by centrifugation (e.g., 1,000×g for 10 minutes). About 20-50 ml per bleed may be obtained from rabbits.

In some aspects, the methods can further comprise the use of IL-2 in the culture. It is understood and herein contemplated that the concentration of the IL-2 can be adjusted to maximize the expansion of TILs. For example, the IL-2 concentration used to culture TILs can be 500, 600, 700, 800, 900, 1000, 1500, 2000, 2500, 3000, 3500, 4000, 4500, 5000, 5500, 6000, 6500, 7000, 7500, 8000 IU/mL or more.

The culture process employed by the art understood methods takes 5-7 weeks to expand TILs from bulk non-purified tumor digests. This is a significant problem in the art as additional time to initiating adoptive transfer therapy of TILs represents an increased risk to the patient due to progression of malignancy while the cell product is being prepared. Moreover, the added time needed for culturing requires additional resources of the hospital in additional personnel to requirements to maintain the culture and costs for media and maintaining a cleanroom. The present method decreases the expansion time to less than 5 weeks resulting in decreased attrition patients from therapy secondary to disease progression. For example, culturing to obtain an expanded population of TILs can occur for any time between 1 day and 5 weeks (35 days), preferably between 21 days (3 weeks) and 5 weeks (35 days), more preferably between 4 weeks (28 days) and 5 weeks (35 days). For example, the culture time can be less than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 days. In some aspect, the pre-REP expansion is harvested when the desired expansion is reached, but not more than 4 weeks. Thus, disclosed herein are TIL expansion methods wherein the pre-REP culture is 1, 2, 3, 4, 5, 6, 7 (1 week), 8, 9, 10, 11, 12, 13, 14 (2 weeks), 15, 16, 17, 18, 19, 20, 21 (3 weeks), 22, 23, 24, 25, 26, 27, or 28 (4 weeks) days. Following the pre-REP, the pre-REP TIL can be frozen and used at a later time. Ultimately, the fresh or thawed pre-REP TIL are submitted to a rapid expansion protocol (REP) which can last less than 1, 2, 3, 4, 5, 6, 7, 8, 9, 10, 11, 12, 13, 14 days. Where frozen TILs are used, the TILs can be thawed for 1-3 days. In some aspect, where thawed TILs are used, and the recovery of the thawed TILS is below 40×10⁶, a second culture of thawed TILs can be used to augment the number of TILs.

To maintain the quality of the nutrients in culture and remove any waste, it is understood and herein contemplated that the all or a portion of the media in the reservoir may be exchanged. The exchange of media can comprise 5, 10, 15, 20, 25, 30, 35, 40, 45, 50, 55, 60, 65, 70, 75, 80, 85, 90, 95, or 100% removal and replacement of media. This media exchange can be accomplished employing any acceptable method for proper tissue culture maintenance known in the art. In one aspect, the media exchange can occur at least one time during the culture of the TILs. For example, the media in the reservoir can be exchanged 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 times during the culture period. That is, the media exchange can occur once during the culture period, once every 15 days, once every 10 days, once every 7 days, once every 5 days, once every other day, or about 2 to 3 times per week.

The culture methods employed herein can utilize any complete media comprising IL-2 appropriate for the growth and propagation of the TILs, including, but not limited to Minimum Essential Medium (MEM), Eagles's Minimum Essential Medium (EMEM), Dulbecco's Minimum Essential Medium (DMEM) Medium 199, RPMI 1640, CMRL-1066, BGJb Medium, Iscove's Modified Dulbecco's Medium (IMDM), and Blood Cell Media.

The TILs can be cultured in any gas permeable reservoir suitable for cell culture and the expansion of TILs. In one aspect, it is understood and herein contemplated that large tissue culture flasks can slow down the expansion of TILs as it takes longer for cells to reach confluency. In one aspect, the gas permeable reservoir can be a tissue culture plate comprising 6 (approximately 10 cm² surface area per well and 60 cm² total surface area), 12(approximately 4 cm² surface area per well and approximately 48 cm² total surface area), 24 (approximately 2 cm² surface area per well and approximately 48 cm² total surface area), 48(approximately 1 cm² surface area per well and approximately 48 cm² total surface area), or 96 (approximately 0.32 cm² surface area per well and 31 cm² total surface area) wells (for example, G-Rex24 well plate or G-Rex6 well plate manufactured by Wilson Wolf). In some aspect, the plates can be silicone coated.

### C. METHODS OF INCREASING THE PROPORTION OF STEM-LIKE T CELLS IN TIL POPULATION

Also disclosed herein are methods of increasing the proportion of Stem-like CD8 T cells in a population of TILs. One problem with traditional adoptive therapy is that the TIL population can become terminally exhausted no longer being able to exert its effector function. As disclosed herein the presently described culture methods can shift the percentage of terminally exhausted T cells to stem-like T cells which have more effector potential. As used herein, “terminally exhausted” T cells refers to T cells classically being CD39+ and CD69+ T cells. The cells can include a higher percentage of one or more of PD-1, CTLA-4, LAG3, TIM-3, TIGIT, BTLA, 2B4, CD160 and a loss or decrease in TCF-1 expression. As used herein “stem-like” refers to T cells that are CD39-CD69-T cells. Stem-like T cells can also be characterized as CCR7+CD45RA+CD95+ or CCR7+CD62L+CD45RA+CD45RO-CD95+. Stem-like cells can also include expression or one or more of CD28, CD27, IL-7Ra, CD11a, IL-2Rb, CD58, CD122, CXCR3, CD31, CD127, and TCF1. Accordingly, in one aspect, disclosed herein are method of increasing the proportion of Stem-like CD8 T cells in a population of TILs in vitro or ex vivo comprising obtaining TILs and culturing the TILs in media comprising one or more CD40 agonists (such as, for example, a CD40 ligand (CD40L) and/or anti-CD40 antibody). In one aspect, the TILs can be cultured for at least 3, 4, 5, 6, 7, 8, 9, 10, 11, 12, 13, 14, 15, 16, 17, 18, 19, 20, 21, 22, 23, 24, 25, 26, 27, 28, 29, 30, or 31 days.

It is understood and herein contemplated that the TILs are obtained from a biopsy (such as, for example, core biopsies and/or one or more surgical resections) of a surgically resectable tumor. In one aspect, the disclosed methods of producing an expanded TIL population comprise obtaining one or more biopsies from the subject (such as, for example, percutaneous tumor samples). As used herein, “biopsy” can include any partial removal of a tissue such as excisional, incisional, core, or fine needle aspiration biopsies. It is understood and herein contemplated that the use of TILs obtained from biopsies (such as, for example, core biopsies including core needle biopsies) makes TIL therapy available to patients who are not eligible for surgery and for patients with unresectable tumors. In addition, core biopsies (such as, for example core needle biopsies) allows for image guided sampling from several anatomical sites.

Where biopsies, and in particular, core biopsies are used as the source of the tissue sample, it is understood and herein contemplated that core biopsies (such as, for example core needle biopsies) can be obtained using any device with which a core biopsy can be obtained (see, for example, the Bard Core Biopsy Instruments and Temno Biopsy Systems by Carefusion such as, BARD MAGNUM®, BARD MAX-CORE®, BARD BIOPTY-CUT®, BARD MARQUEE®, BARD MISSION®, and BARD MONOPTY® from CR Bard, Inc.).

The needle for obtaining the biopsy can be 6, 8, 10, 12, 14, 16, 18, or 20 gauge needle with a needle length between about 2 cm and to about 30 cm long, preferably between about 10 cm and about 25 cm long, more preferably between about 16 cm and about 20 cm long. For example, the needle length for obtaining a core biopsy can be 2, 3, 4, 5, 6, 7, 8, 9, 10, 11, 12, 13, 14, 15, 16, 17, 18, 19, 20, 21, 22, 23, 24, 25, 26, 27, 28, 29, or 30 cm long. The penetration depth of the needle can be between about 15 mm and 30 mm, preferably between about 20 mm and 25 mm. For example, the penetration depth can be 15, 16, 17, 18, 19, 20, 21, 22, 23, 24, 25, 26, 27, 28, 29, or 30 mm.

In one aspect, the use of core biopsy allows the ability to target certain and possibly multiple areas of a tumor. In one aspect, disclosed herein are methods of increasing the proportion of Stem-like CD8 T cells in a population of TILs further comprising the use of imaging techniques such as radiomics to guide TIL acquisition.

Once obtained, tissue samples, including, but not limited to biopsies (such as, for example, core biopsies including core needle biopsies) and/or surgical resections provide the added advantage of not requiring further sectioning (i.e., making fragments), but can be directly digested. In one aspect, the disclosed methods can comprise placing the tissue sample directly into a digesting solution (such as, for example a at least one, or a combination of two, or all three of the human or humanized enzymes selected from the group consisting of collagenase (e.g., XIAFLEX®), hyaluronidase (e.g., HYLENEX®), and DNAse (e.g., PULMOZYME®)).

The tumor cells can be dissociated using mechanical fragmentation (either pooled fragments or separated) and/or using a tumor digest. Accordingly, disclosed herein are methods of increasing the proportion of Stem-like CD8 T cells in a population of TILs, wherein the one or more core biopsies are digested directly from the patient without disaggregation of the specimen. Digests are well known in the art and typically comprise a collagenase, hyaluronidase, and DNAse. It is understood and herein contemplated that the digest can comprise at least one, or a combination of two, or all three of the human or humanized enzymes selected from the group consisting of collagenase (e.g., XIAFLEX®), hyaluronidase (e.g., HYLENEX®), and DNAse (e.g., PULMOZYME®). As noted above, in some instances the digest can occur directly from the tissue sample without prior fragmentation (mechanical or otherwise) or the separation or pooling of fragments thereby creating a bulk non-purified digest.

The concentration of fragmented and/or digested cells used in the pre-REP of the disclosed methods can affect the yield and or efficacy of the disclosed methods. In one aspect, the methods utilizes less than 5×10⁶ cells, for example, the method can use 4×10⁶, 3×10⁶, 2×10⁶, 1×10⁶, 9×10⁵, 8×10⁵, 7×10⁵, 6×10⁵, 5×10⁵, 4×10⁵, 3×10⁵, 2×10⁵, or 1×10⁵ or less bulk non-purified digest cells per tissue culture well.

Also disclosed herein are methods of increasing the proportion of Stem-like CD8 T cells in a population of TILs, further adding autologous B cells to the culture of TILs and CD40L.

In some aspects, the methods can further comprise the use of IL-2 in the culture. It is understood and herein contemplated that the concentration of the IL-2 can be adjusted to maximize the expansion of TILs. For example, the IL-2 concentration used to culture TILs can be 500, 600, 700, 800, 900, 1000, 1500, 2000, 2500, 3000, 3500, 4000, 4500, 5000, 5500, 6000, 6500, 7000, 7500, 8000 IU/mL or more.

The culture process employed by the art understood methods takes 5-7 weeks to expand TILs from bulk non-purified tumor digests. This is a significant problem in the art as additional time to initiating adoptive transfer therapy of TILs represents an increased risk to the patient due to progression of malignancy while the cell product is being prepared. Moreover, the added time needed for culturing requires additional resources of the hospital in additional personnel to requirements to maintain the culture and costs for media and maintaining a cleanroom. The present method decreases the expansion time to less than 5 weeks resulting in decreased attrition patients from therapy secondary to disease progression. For example, culturing to obtain an expanded population of TILs can occur for any time between 1 day and 5 weeks (35 days), preferably between 21 days (3 weeks) and 5 weeks (35 days), more preferably between 4 weeks (28 days) and 5 weeks (35 days). For example, the culture time can be less than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 days. In some aspect, the pre-REP expansion is harvested when the desired expansion is reached, but not more than 4 weeks. Thus, disclosed herein are TIL expansion methods wherein the pre-REP culture is 1, 2, 3, 4, 5, 6, 7 (1 week), 8, 9, 10, 11, 12, 13, 14 (2 weeks), 15, 16, 17, 18, 19, 20, 21 (3 weeks), 22, 23, 24, 25, 26, 27, or 28 (4 weeks) days. Following the pre-REP, the pre-REP TIL can be frozen and used at a later time. Ultimately, the fresh or thawed pre-REP TIL are submitted to a rapid expansion protocol (REP) which can last less than 1, 2, 3, 4, 5, 6, 7, 8, 9, 10, 11, 12, 13, 14 days. Where frozen TILs are used, the TILs can be thawed for 1-3 days. In some aspect, where thawed TILs are used, and the recovery of the thawed TILS is below 40×10⁶, a second culture of thawed TILs can be used to augment the number of TILs.

To maintain the quality of the nutrients in culture and remove any waste, it is understood and herein contemplated that the all or a portion of the media in the reservoir may be exchanged. The exchange of media can comprise 5, 10, 15, 20, 25, 30, 35, 40, 45, 50, 55, 60, 65, 70, 75, 80, 85, 90, 95, or 100% removal and replacement of media. This media exchange can be accomplished employing any acceptable method for proper tissue culture maintenance known in the art. In one aspect, the media exchange can occur at least one time during the culture of the TILs. For example, the media in the reservoir can be exchanged 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 times during the culture period. That is, the media exchange can occur once during the culture period, once every 15 days, once every 10 days, once every 7 days, once every 5 days, once every other day, or about 2 to 3 times per week.

The culture methods employed herein can utilize any complete media comprising IL-2 appropriate for the growth and propagation of the TILs, including, but not limited to Minimum Essential Medium (MEM), Eagles's Minimum Essential Medium (EMEM), Dulbecco's Minimum Essential Medium (DMEM) Medium 199, RPMI 1640, CMRL-1066, BGJb Medium, Iscove's Modified Dulbecco's Medium (IMDM), and Blood Cell Media.

The TILs can be cultured in any gas permeable reservoir suitable for cell culture and the expansion of TILs. In one aspect, it is understood and herein contemplated that large tissue culture flasks can slow down the expansion of TILs as it takes longer for cells to reach confluency. In one aspect, the gas permeable reservoir can be a tissue culture plate comprising 6 (approximately 10 cm² surface area per well and 60 cm² total surface area), 12(approximately 4 cm² surface area per well and approximately 48 cm² total surface area), 24 (approximately 2 cm² surface area per well and approximately 48 cm² total surface area), 48(approximately 1 cm² surface area per well and approximately 48 cm² total surface area), or 96 (approximately 0.32 cm² surface area per well and 31 cm² total surface area) wells (for example, G-Rex24 well plate or G-Rex6 well plate manufactured by Wilson Wolf). In some aspect, the plates can be silicone coated.

### D. ANTI-CD40 ANTIBODIES

To date, a variety of agonistic anti-CD40 mAbs are currently under investigation in clinical trials, as monotherapies or in combination with other agents.

CP-870,893 (now licensed to Roche Diagnostics under the names R07009789 or Selicrelumab) is a fully humanized monoclonal IgG2 antibody that binds CD40 with a very high affinity (Kd of 0.4 nmol/1) (66,67). CP-870,893 has been shown in preclinical studies to be a strong agonist of CD40 without eliciting antibody-dependent cellmediated cytotoxicity (ADCC), a mechanism through which an antibody induces target lysis by activating host leukocytic effector cells, or complement dependent cytotoxicity, a cascade of complement-related reactions leading to target lysis.

A study was conducted in 22 patients who had chemotherapy-naive advanced pancreatic ductal adenocarcinoma (PDA). The combination of a 0.2 mg/kg dose of CP-870,893 every 3 weeks and standard-of-care gemcitabine was well tolerated in the subjects. The objective response rate (ORR) was 19%, the progression-free survival (PFS) was 5.2 months, and median overall survival was 8.4 months. With FDG-PET/CT imaging guidance, the authors found that some lesions responded and others failed to respond during therapy, suggesting that treatment responses to this therapy were heterogeneous.

Dacetuzumab, also named SEA-40 or SGN-40, is a humanized CD40 targeted IgG1 mAb developed by Seattle Genetics, Inc. As a weak agonist (Kd nmol/1), dacetuzumab does not block the CD40/CD40L interaction in vitro. Dacetuzumab was engineered in an afucosylated IgG1 format to improve the ADCC potential. Preclinical results have demonstrated that dacetuzumab induces apoptosis of non-Hodgkin's lymphoma cells in vivo by ADCC, antibody-dependent cellular phagocytosis (ADCP), and direct apoptotic signaling.

In a pilot phase Ib study, a regimen of dacetuzumab combined with rituximab and gemcitabine was investigated in patients with relapsed or refractory DLBCLs. The complete response rate in this study was 20%, and the partial response rate was 27%. Due to this efficacy outcome, a randomized, double-blind, placebo-controlled, phase IIb clinical trial was conducted to investigate dacetuzumab or placebo in combination with rituximab plus ifosfamide, carboplatin, and etoposide chemotherapy in 151 patients with relapsed or refractory DLBCL

ChiLob 7/4 (University of Southampton, UK) is a chimeric agonistic anti-CD40 IgG1 antibody. Preclinical studies showed that ChiLob 7/4 has the ability to inhibit the growth of various CD40-expressing human malignant lymphoma and epithelial cell lines.

ADC-1013, sponsored by Alligator Bioscience, is a fully human agonistic anti-CD40 IgG1 mAb with high affinity for CD40 (Kd=0.01 nM).

CDX-1140, developed by Celldex Therapeutics, Inc., is a human IgG2 antibody that stimulates CD40 signalling without the requirement for cross-linking or Fc receptor interactions.

ABBV-927 (AbbVie, Inc.) is an anti-CD40/anti-mesothelin bispecific antibody that is being tested in phase I trials for the treatment of advanced solid tumours, including non-small cell lung cancer, squamous cell carcinoma of the head and neck, cutaneous malignant melanoma, and pancreatic adenocarcinoma, as monotherapy or in combination with other immunotherapies (anti-PD-1 and anti-OX40 antibodies).

APX005M, developed by Apexigen, is a humanized mAb IgG1/k against CD40. A preclinical study has demonstrated that APX005M binds to CD40 at the CD40L binding domain with a high affinity in mice (Kd=0.12 nM) and monkeys (Kd=0.37 nM). U.S. Pat. No. 8,088,383 describes methods for treating B-cell malignancies using antagonist anti-CD40 antibodies, which is incorporated herein for the teaching of these antibodies and methods.

The monoclonal antibody 15B8 represents a suitable antagonist anti-CD40 antibody for use in the methods of the present invention. This antibody is described in U.S. Provisional Application Ser. No. 60/237,556, titled “Human Anti-CD40 Antibodies,” filed Oct. 2, 2000, and PCT International Application No. PCT/US01/30857, also titled “Human Anti-CD40 Antibodies,” filed Oct. 2, 2001 (Attorney Docket No. PP 16092.003), both of which are herein incorporated by reference in their entirety. The 15B8 antibody is a fully human anti-CD40 monoclonal antibody of the IgG2 isotype produced from the hybridoma cell line 15B8. The cell line was created using splenocytes from an immunized xenotypic mouse containing a human immunoglobulin locus (Abgenix). The spleen cells were fused with the mouse myeloma SP2/0 cells (Sierra BioSource). The resulting hybridomas were sub-cloned several times to create the stable monoclonal cell line 15B8. The hybridoma cell line 15B8 was deposited with the American Type Culture Collection (ATCC), 10801 University Boulevard, Manassas, Va., USA, on Oct. 25, 2001, under the terms of the Budapest Treaty and assigned Patent Deposit— Designation PTA-3814.

The 15B8 cell line was adapted to grow in protein-free medium and used to create a Master Cell Bank. The Master Cell Bank was tested for identity and adventitious and endogenous contaminants. The Master Cell Bank was used to manufacture the desired human IgG2. The respective 15B8 antibody was purified using chromatography and filtration procedures.

The anti-CD40 antibody 15B8 is a polypeptide composed of 1,284 amino acid residues with a predicted molecular weight of 149,755 with two heavy chains and two light chains in a heterodimeric arrangement. Amino acid analysis reveals that the antibody is composed of equimolar amounts of heavy and light chains. The nucleotide and amino acid sequences for the variable region for the light chain are set forth in SEQ ID NO:1 and SEQ ID NO:2, respectively. The nucleotide and amino acid sequences for the variable region for the heavy chain are set forth in SEQ ID NO:3 and SEQ ID NO:4, respectively. The 15B8 monoclonal antibody binds soluble CD40 in ELISA-type assays. When tested in vitro for effects on proliferation of B cells from numerous primates, 15B8 acts as an agonistic anti-CD40 antibody in cynomologus, baboon, and rhesus monkeys. In assays with humans, chimpanzees, and marmosets, 15B8 is an antagonist anti-CD40 antibody. The binding affinity of 15B8 to human CD40 is 3.1×10⁻⁹ M as determined by the Biacore™ assay.

Suitable antagonist anti-CD40 antibodies for use in the methods of the present invention exhibit a strong single-site binding affinity for the CD40 cell-surface antigen. The monoclonal antibodies of the invention exhibit a dissociation constant (Kd) for CD40 of at least 10⁻⁵ M, at least 3×10⁻⁵ M, preferably at least 10⁻⁶ M to 10⁻⁷ M, more preferably at least 10−8 M to about 10−20 M, yet more preferably at least 5×10⁻⁹ M to about 10⁻¹⁸ M, most preferably at least about 5×10⁻⁹ M to about 10−16 M, such as 10⁻⁸ M, 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, 10⁻¹³ M, 5×10⁻¹⁴ M, 10⁻¹⁴ M, 5×10⁻¹⁵ M, 10⁻¹⁵ M, 5×10⁻¹⁶ M, or 10⁻¹⁶ M, as measured using a standard assay such as Biacore™.

Biacore™ analysis is known in the art and details are provided in the “BIAapplications handbook.”

a) Antibodies Generally

The term “antibodies” is used herein in a broad sense and includes both polyclonal and monoclonal antibodies. In addition to intact immunoglobulin molecules, also included in the term “antibodies” are fragments or polymers of those immunoglobulin molecules, and human or humanized versions of immunoglobulin molecules or fragments thereof, as long as they are chosen for their ability to interact with CD40 such that CD40 is inhibited from interacting with CD40L. The antibodies can be tested for their desired activity using the in vitro assays described herein, or by analogous methods, after which their in vivo therapeutic and/or prophylactic activities are tested according to known clinical testing methods. There are five major classes of human immunoglobulins: IgA, IgD, IgE, IgG and IgM, and several of these may be further divided into subclasses (isotypes), e.g., IgG-1, IgG-2, IgG-3, and IgG-4; IgA-1 and IgA-2. One skilled in the art would recognize the comparable classes for mouse. The heavy chain constant domains that correspond to the different classes of immunoglobulins are called alpha, delta, epsilon, gamma, and mu, respectively.

The term “monoclonal antibody” as used herein refers to an antibody obtained from a substantially homogeneous population of antibodies, i.e., the individual antibodies within the population are identical except for possible naturally occurring mutations that may be present in a small subset of the antibody molecules. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, as long as they exhibit the desired antagonistic activity.

The disclosed monoclonal antibodies can be made using any procedure which produces mono clonal antibodies. For example, disclosed monoclonal antibodies can be prepared using hybridoma methods, such as those described by Kohler and Milstein, Nature, 256:495 (1975). In a hybridoma method, a mouse or other appropriate host animal is typically immunized with an immunizing agent to elicit lymphocytes that produce or are capable of producing antibodies that will specifically bind to the immunizing agent. Alternatively, the lymphocytes may be immunized in vitro.

The monoclonal antibodies may also be made by recombinant DNA methods. DNA encoding the disclosed monoclonal antibodies can be readily isolated and sequenced using conventional procedures (e.g., by using oligonucleotide probes that are capable of binding specifically to genes encoding the heavy and light chains of murine antibodies). Libraries of antibodies or active antibody fragments can also be generated and screened using phage display techniques, e.g., as described in U.S. Pat. No. 5,804,440 to Burton et al. and U.S. Pat. No. 6,096,441 to Barbas et al.

In vitro methods are also suitable for preparing monovalent antibodies. Digestion of antibodies to produce fragments thereof, particularly, Fab fragments, can be accomplished using routine techniques known in the art. For instance, digestion can be performed using papain. Examples of papain digestion are described in WO 94/29348 published Dec. 22, 1994 and U.S. Pat. No. 4,342,566. Papain digestion of antibodies typically produces two identical antigen binding fragments, called Fab fragments, each with a single antigen binding site, and a residual Fc fragment. Pepsin treatment yields a fragment that has two antigen combining sites and is still capable of cross-linking antigen.

As used herein, the term “antibody or fragments thereof” encompasses chimeric antibodies and hybrid antibodies, with dual or multiple antigen or epitope specificities, and fragments, such as F(ab′)2, Fab′, Fab, Fv, scFv, VHH, and the like, including hybrid fragments. Thus, fragments of the antibodies that retain the ability to bind their specific antigens are provided. For example, fragments of antibodies which maintain CD40 binding activity are included within the meaning of the term “antibody or fragment thereof” Such antibodies and fragments can be made by techniques known in the art and can be screened for specificity and activity according to the methods set forth in the Examples and in general methods for producing antibodies and screening antibodies for specificity and activity (See Harlow and Lane. Antibodies, A Laboratory Manual. Cold Spring Harbor Publications, New York, (1988)).

Also included within the meaning of “antibody or fragments thereof” are conjugates of antibody fragments and antigen binding proteins (single chain antibodies).

The fragments, whether attached to other sequences or not, can also include insertions, deletions, substitutions, or other selected modifications of particular regions or specific amino acids residues, provided the activity of the antibody or antibody fragment is not significantly altered or impaired compared to the non-modified antibody or antibody fragment. These modifications can provide for some additional property, such as to remove/add amino acids capable of disulfide bonding, to increase its bio-longevity, to alter its secretory characteristics, etc. In any case, the antibody or antibody fragment must possess a bioactive property, such as specific binding to its cognate antigen. Functional or active regions of the antibody or antibody fragment may be identified by mutagenesis of a specific region of the protein, followed by expression and testing of the expressed polypeptide. Such methods are readily apparent to a skilled practitioner in the art and can include site-specific mutagenesis of the nucleic acid encoding the antibody or antibody fragment. (Zoller, M. J. Curr. Opin. Biotechnol. 3:348-354, 1992).

Fragments of the anti-CD40 antibodies are suitable for use in the methods of the invention so long as they retain the desired affinity of the full-length antibody. Thus, a fragment of an anti-CD40 antibody will retain the ability to bind to the CD40 B-cell surface antigen. Such fragments are characterized by properties similar to the corresponding full-length antagonist anti-CD40 antibody, that is the fragments will 1) specifically bind a human CD40 antigen expressed on the surface of a human cell; 2) are free of significant agonist activity when bound to a CD40 antigen on a normal human B cell; and 3) exhibit antagonist activity when bound to a CD40 antigen on a malignant human B cell. Where the full-length antagonist anti-CD40 antibody exhibits antagonist activity when bound to the CD40 antigen on the surface of a normal human B cell, the fragment will also exhibit such antagonist activity. Such fragments are referred to herein as “antigen-binding” fragments.

As used herein, the term “antibody” or “antibodies” can also refer to a human antibody and/or a humanized antibody. Many non-human antibodies (e.g., those derived from mice, rats, or rabbits) are naturally antigenic in humans, and thus can give rise to undesirable immune responses when administered to humans. Therefore, the use of human or humanized antibodies in the methods serves to lessen the chance that an antibody administered to a human will evoke an undesirable immune response.

Further, an antibody (or fragment thereof) may be conjugated to a therapeutic moiety such as a cytotoxin, a therapeutic agent, or a radioactive metal ion. A cytotoxin or cytotoxic agent includes any agent that is detrimental to cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine). The conjugates of the invention can be used for modifying a given biological response; the drug moiety is not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, interferon-alpha, interferon-beta, nerve growth factor, platelet derived growth factor, tissue plasminogen activator; or, biological response modifiers such as, for example, lymphokines, interleukin-1 (“IL-1”), interleukin-2 (“IL-2”), interleukin-6 (“IL-6”), granulocyte macrophase colony stimulating factor (“GM-CSF”), granulocyte colony stimulating factor (“G-CSF”), or other growth factors.

Additionally, the term “anti-CD40 antibody” as used herein encompasses chimeric anti-CD40 antibodies. By “chimeric” antibodies is intended antibodies that are most preferably derived using recombinant deoxyribonucleic acid techniques and which comprise both human (including immunologically “related” species, e.g., chimpanzee) and non-human components. Thus, the constant region of the chimeric antibody is most preferably substantially identical to the constant region of a natural human antibody; the variable region of the chimeric antibody is most preferably derived from a non-human source and has the desired antigenic specificity to the CD40 cell-surface antigen. The non-human source can be any vertebrate source that can be used to generate antibodies to a human CD40 cell-surface antigen or material comprising a human CD40 cell-surface antigen. Such non-human sources include, but are not limited to, rodents (e.g., rabbit, rat, mouse, etc.; see, for example, U.S. Pat. No. 4,816,567, herein incorporated by reference) and non-human primates (e.g., Old World Monkey, Ape, etc.; see, for example, U.S. Pat. Nos. 5,750,105 and 5,756,096; herein incorporated by reference). As used herein, the phrase “immunologically active” when used in reference to chimeric anti-CD40 antibodies means a chimeric antibody that binds human CD40.

b) Human Antibodies

The disclosed human antibodies can be prepared using any technique. The disclosed human antibodies can also be obtained from transgenic animals. For example, transgenic, mutant mice that are capable of producing a full repertoire of human antibodies, in response to immunization, have been described (see, e.g., Jakobovits et al., Proc. Natl. Acad. Sci. USA, 90:2551-255 (1993); Jakobovits et al., Nature, 362:255-258 (1993); Bruggermann et al., Year in Immunol., 7:33 (1993)). Specifically, the homozygous deletion of the antibody heavy chain joining region (J(H)) gene in these chimeric and germ-line mutant mice results in complete inhibition of endogenous antibody production, and the successful transfer of the human germ-line antibody gene array into such germ-line mutant mice results in the production of human antibodies upon antigen challenge. Antibodies having the desired activity are selected using Env-CD4-co-receptor complexes as described herein.

c) Humanized Antibodies

Humanized anti-CD40 antibodies are also encompassed by the term anti-CD40 antibody as used herein. By “humanized” is intended forms of anti-CD40 antibodies that contain minimal sequence derived from non-human immunoglobulin sequences. Antibody humanization techniques generally involve the use of recombinant DNA technology to manipulate the DNA sequence encoding one or more polypeptide chains of an antibody molecule. Accordingly, a humanized form of a non-human antibody (or a fragment thereof) is a chimeric antibody or antibody chain (or a fragment thereof, such as an sFv, Fv, Fab, Fab′, F(ab′)2, or other antigen-binding portion of an antibody) which contains a portion of an antigen binding site from a non-human (donor) antibody integrated into the framework of a human (recipient) antibody.

To generate a humanized antibody, residues from one or more complementarity determining regions (CDRs) of a recipient (human) antibody molecule are replaced by residues from one or more CDRs of a donor (non-human) antibody molecule that is known to have desired antigen binding characteristics (e.g., a certain level of specificity and affinity for the target antigen). In some instances, Fv framework (FR) residues of the human antibody are replaced by corresponding non-human residues. Humanized antibodies may also contain residues which are found neither in the recipient antibody nor in the imported CDR or framework sequences. Generally, a humanized antibody has one or more amino acid residues introduced into it from a source which is non-human. In practice, humanized antibodies are typically human antibodies in which some CDR residues and possibly some FR residues are substituted by residues from analogous sites in rodent antibodies. Humanized antibodies generally contain at least a portion of an antibody constant region (Fc), typically that of a human antibody (Jones et al., Nature, 321:522-525 (1986), Reichmann et al., Nature, 332:323-327 (1988), and Presta, Curr. Opin. Struct. Biol., 2:593-596 (1992)).

Methods for humanizing non-human antibodies are well known in the art. For example, humanized antibodies can be generated according to the methods of Winter and co-workers (Jones et al., Nature, 321:522-525 (1986), Riechmann et al., Nature, 332:323-327 (1988), Verhoeyen et al., Science, 239:1534-1536 (1988)), by substituting rodent CDRs or CDR sequences for the corresponding sequences of a human antibody. Methods that can be used to produce humanized antibodies are also described in U.S. Pat. No. 4,816,567 (Cabilly et al.), U.S. Pat. No. 5,565,332 (Hoogenboom et al.), U.S. Pat. No. 5,721,367 (Kay et al.), U.S. Pat. No. 5,837,243 (Deo et al.), U.S. Pat. No. 5,939,598 (Kucherlapati et al.), U.S. Pat. No. 6,130,364 (Jakobovits et al.), and U.S. Pat. No. 6,180,377 (Morgan et al.).

d) Administration of Antibodies

Administration of the antibodies can be done as disclosed herein. Nucleic acid approaches for antibody delivery also exist. The broadly neutralizing anti-CD40 antibodies and antibody fragments can also be administered to patients or subjects as a nucleic acid preparation (e.g., DNA or RNA) that encodes the antibody or antibody fragment, such that the patient's or subject's own cells take up the nucleic acid and produce and secrete the encoded antibody or antibody fragment. The delivery of the nucleic acid can be by any means, as disclosed herein, for example.

Antagonist anti-CD40 antibodies useful in the methods of the present invention include the 15B8 monoclonal antibody disclosed herein as well as antibodies differing from this antibody but retaining the CDRs; and antibodies with one or more amino acid addition(s), deletion(s), or substitution(s), wherein the antagonist activity is measured by inhibition of malignant B cell proliferation and/or differentiation. The invention also encompasses de-immunized antagonist anti-CD40 antibodies, which can be produced as described in, for example, International Publication Nos. WO 98/52976 and WO 0034317; herein incorporated by reference. In this manner, residues within the antagonist anti-CD40 antibodies of the invention are modified so as to render the antibodies non- or less immunogenic to humans while retaining their antagonist activity toward malignant human B cells, wherein such activity is measured by assays noted elsewhere herein. Also included within the scope of the claims are fusion proteins comprising an antagonist anti-CD40 antibody of the invention, or a fragment thereof, which fusion proteins can be synthesized or expressed from corresponding polynucleotide vectors, as is known in the art. Such fusion proteins are described with reference to conjugation of antibodies as noted below.

The antibodies of the present invention can have sequence variations produced using methods described in, for example, Patent Publication Nos. EP 0 983 303 A1, WO 00/34317, and WO 98/52976, incorporated herein by reference. For example, it has been shown that sequences within the CDR can cause an antibody to bind to MHC Class II and trigger an unwanted helper T cell response. A conservative substitution can allow the antibody to retain binding activity yet lose its ability to trigger an unwanted T cell response. Any such conservative or non-conservative substitutions can be made using art-recognized methods, such as those noted elsewhere herein, and the resulting antibodies will fall within the scope of the invention. The variant antibodies can be routinely tested for antagonist activity, affinity, and specificity using methods described herein.

### E. CHEMOKINE GENE SIGNATURE

Disclosed herein is a method for predicting the responsiveness of a subject to CD40 agonist therapy, such as agonist anti-CD40 therapy, comprising assaying a tumor sample from the subject for a 12 Chemokine gene signature demonstrating that the tumor comprises tertiary lymphoid structures containing B cells.

Chemokines, which are small protein molecules involved in immune and inflammatory responses, direct leukocyte trafficking to areas of injury as well as to locations where primary immune responses are initiated (secondary lymphoid tissues such as lymph nodes, spleen, Peyer's patches, and tonsils). There are presently four classes of chemokine molecules (C, CC, CXC, and CX3C) that are named for the number and location of cysteine residues on the amino terminus of the protein. These molecules communicate with their target cells via G-protein coupled receptors that are pertussis toxin sensitive. Different chemokines act on different leukocyte populations, thereby modulating the influx of immune effector cells to the area in question based on the needs of the particular situation. Chemokines are secreted proteins involved in immunoregulatory and inflammatory processes. The chemokines of the disclosed gene signature are shown in Table 1.

TABLE 1
Chemokines

| Gene | | GenBank Acc. No: | GenBank Acc. No: |
|---|---|---|---|
| Symbol | Gene Name | Nucleic Acid | Amino Acid |
| CCL2 | chemokine (C-C motif) ligand 2 | NM_002982.3 | NP_002973.1 |
| CCL3 | chemokine (C-C motif) ligand 3 | NM_002983.2 | NP_002974.1 |
| CCL4 | chemokine (C-C motif) ligand 4 | NM_002984.2 | NP_002975.1 |
| CCL5 | chemokine (C-C motif) ligand 5 | NM_002985.2 | NP_002976.2 |
| CCL8 | chemokine (C-C motif) ligand 8 | NM_005623.2 | NP_005614.2 |
| CCL18 | chemokine (C-C motif) ligand 18 | NM_002988.2 | NM_002988.2 |
| | (pulmonary and activation-regulated) | | |
| CCL19 | chemokine (C-C motif) ligand 19 | NM_006274.2 | NP_006265.1 |
| CCL21 | chemokine (C-C motif) ligand 21 | NM_002989.2 | NP_002980.1 |
| CXCL9 | chemokine (C-X-C motif) ligand 9 | NM_002416.1 | NP_002407.1 |
| CXCL10 | chemokine (C-X-C motif) ligand 10 | NM_001565.2 | NP_001556.2 |
| CXCL11 | chemokine (C-X-C motif) ligand 11 | NM_005409.4 | NP_005400.1 |
| CXCL13 | chemokine (C-X-C motif) ligand 13 | NM_006419.2 | NP_006410.1 |

In some embodiments, the methods include assaying the presence or levels of chemokine mRNA or proteins in the sample. The presence and/or level of a protein can be evaluated using methods known in the art, e.g., using quantitative immunoassay methods. The presence and/or level of an mRNA can be evaluated using methods known in the art, e.g., Northern blotting or quantitative PCR methods, e.g., RT-PCR. In some embodiments, high throughput methods, e.g., protein or gene chips as are known in the art (see, e.g., Ch. 12, Genomics, in Griffiths et al., Eds. Modern genetic Analysis, 1999, W. H. Freeman and Company; Ekins and Chu, Trends in Biotechnology, 1999, 17:217-218; MacBeath and Schreiber, Science 2000, 289(5485):1760-1763; Simpson, Proteins and Proteomics: A Laboratory Manual, Cold Spring Harbor Laboratory Press; 2002; Hardiman, Microarrays Methods and Applications: Nuts & Bolts, DNA Press, 2003), can be used to detect the presence and/or level of chemokine proteins as described herein.

In some embodiments, the methods include assaying levels of one or more control genes or proteins, and comparing the level of expression of the chemokine genes or proteins to the level of the control genes or proteins, to normalize the levels of the chemokine genes or proteins. Suitable endogenous control genes includes a gene whose expression level should not differ between samples, such as a housekeeping or maintenance gene, e.g., 18S ribosomal RNA; beta Actin; Glyceraldehyde-3-phosphate dehydrogenase; Phosphoglycerate kinase 1; Peptidylprolyl isomerase A (cyclophilin A); Ribosomal protein L13a; large Ribosomal protein P0; Beta-2-microglobulin; Tyrosine 3-monooxygenase/tryptophan 5-monooxygenase activation protein, zeta polypeptide; Succinate dehydrogenase; Transferrin receptor (p90, CD71); Aminolevulinate, delta-, synthase 1; Glucuronidase, beta; Hydroxymethyl-bilane synthase; Hypoxanthine phosphoribosyltransferase 1; TATA box binding protein; and/or Tubulin, beta polypeptide.

Generally speaking, the methods described herein can be performed on cells from a tumor. The cells can be obtained by known methods, e.g., during a biopsy (such as a core needle biopsy), or during a surgical procedure to remove all or part of the tumor. The cells can be used fresh, frozen, fixed, and/or preserved, so long as the mRNA or protein that is to be assayed is maintained in a sufficiently intact state to allow accurate analysis.

In some embodiments of the methods described herein, the levels of the chemokine genes in the tumor sample can be compared individually to levels in a reference. The reference levels can represent levels in a tumor that does not have tertiary lymphoid structures (TLSs). Alternatively, reference levels can represent levels in a tumor that does shave TLSs. In some embodiments, the reference levels represent a threshold.

In some embodiments of the methods described herein, values representing the levels of the chemokine genes can be summed to produce a “chemokine gene score” that can be compared to a reference chemokine gene score, wherein a chemokine gene score that is above the reference chemokine gene score indicates that the tumor will produce TILs with enhanced tumor reactivity, and an chemokine gene score below the reference score indicates that the tumor will produce TILs that do not have enhanced tumor reactivity.

For example, in some embodiments, the expression levels of each of the evaluated genes can be assigned a value (e.g., a value that represents the expression level of the gene, e.g., normalized to an endogenous control gene as described herein). That value (optionally weighted to increase or decrease its effect on the final score) can be summed to produce an immune-related gene score. One of skill in the art could optimize such a method to determine an optimal algorithm for determining an immunerelated gene score. The methods described herein can include determining levels (or scores) for all of the 12 chemokines. In some embodiments all of the genes are evaluated, but in some embodiments a subset of one or all of the sets is evaluated.

### F. THERAPEUTIC METHODS

In one aspect, disclosed herein are methods of treating, inhibiting, reducing, decreasing, ameliorating, and/or preventing a cancer and/or metastasis in a subject comprising obtaining tumor infiltrating lymphocytes (TILs) from the subject; culturing the TILs in media comprising one or more CD40 agonists, and administering the cultured TILs to the subject. In one aspect, the TILs can be cultured for at least 3, 4, 5, 6, 7, 8, 9, 10, 11, 12, 13, 14, 15, 16, 17, 18, 19, 20, 21, 22, 23, 24, 25, 26, 27, 28, 29, 30, or 31 days.

It is understood and herein contemplated that the TILs are obtained from a biopsy (such as, for example, core biopsies and/or one or more surgical resections) of a surgically resectable tumor. In one aspect, the disclosed methods of producing an expanded TIL population comprise obtaining one or more biopsies from the subject (such as, for example, percutaneous tumor samples). As used herein, “biopsy” can include any partial removal of a tissue such as excisional, incisional, core, or fine needle aspiration biopsies. It is understood and herein contemplated that the use of TILs obtained from biopsies (such as, for example, core biopsies including core needle biopsies) makes TIL therapy available to patients who are not eligible for surgery and for patients with unresectable tumors. In addition, core biopsies (such as, for example core needle biopsies) allows for image guided sampling from several anatomical sites.

Where biopsies, and in particular, core biopsies are used as the source of the tissue sample, it is understood and herein contemplated that core biopsies (such as, for example core needle biopsies) can be obtained using any device with which a core biopsy can be obtained (see, for example, the Bard Core Biopsy Instruments and Temno Biopsy Systems by Carefusion such as, BARD MAGNUM®, BARD MAX-CORE®, BARD BIOPTY-CUT®, BARD MARQUEE®, BARD MISSION®, and BARD MONOPTY® from CR Bard, Inc.). The needle for obtaining the biopsy can be 6, 8, 10, 12, 14, 16, 18, or 20 gauge needle with a needle length between about 2 cm and to about 30 cm long, preferably between about 10 cm and about 25 cm long, more preferably between about 16 cm and about 20 cm long. For example, the needle length for obtaining a core biopsy can be 2, 3, 4, 5, 6, 7, 8, 9, 10, 11, 12, 13, 14, 15, 16, 17, 18, 19, 20, 21, 22, 23, 24, 25, 26, 27, 28, 29, or 30 cm long. The penetration depth of the needle can be between about 15 mm and 30 mm, preferably between about 20 mm and 25 mm. For example, the penetration depth can be 15, 16, 17, 18, 19, 20, 21, 22, 23, 24, 25, 26, 27, 28, 29, or 30 mm.

In one aspect, the use of core biopsy allows the ability to target certain and possibly multiple areas of a tumor. In one aspect, disclosed herein are methods of treating, inhibiting, reducing, decreasing, ameliorating, and/or preventing a cancer and/or metastasis further comprising the use of imaging techniques such as radiomics to guide TIL acquisition.

Once obtained, tissue samples, including, but not limited to biopsies (such as, for example, core biopsies including core needle biopsies) and/or surgical resections provide the added advantage of not requiring further sectioning (i.e., making fragments), but can be directly digested. In one aspect, the disclosed methods can comprise placing the tissue sample directly into a digesting solution (such as, for example a at least one, or a combination of two, or all three of the human or humanized enzymes selected from the group consisting of collagenase (e.g., XIAFLEX®), hyaluronidase (e.g., HYLENEX®), and DNAse (e.g., PULMOZYME®)).

The tumor cells can be dissociated using mechanical fragmentation (either pooled fragments or separated) and/or using a tumor digest. Accordingly, disclosed herein are methods of expanding tumor infiltrating lymphocytes (TILs), wherein the one or more core biopsies are digested directly from the patient without disaggregation of the specimen. Digests are well known in the art and typically comprise a collagenase, hyaluronidase, and DNAse. It is understood and herein contemplated that the digest can comprise at least one, or a combination of two, or all three of the human or humanized enzymes selected from the group consisting of collagenase (e.g., XIAFLEX®), hyaluronidase (e.g., HYLENEX®), and DNAse (e.g., PULMOZYME®). As noted above, in some instances the digest can occur directly from the tissue sample without prior fragmentation (mechanical or otherwise) or the separation or pooling of fragments thereby creating a bulk non-purified digest.

The concentration of fragmented and/or digested cells used in the pre-REP of the disclosed methods can affect the yield and or efficacy of the disclosed methods. In one aspect, the methods utilizes less than 5×10⁶ cells, for example, the method can use 4×10₆, 3×10⁶, 2×10⁶, 1×10⁶, 9×10⁵, 8×10⁵, 7×10⁵, 6×10⁵, 5×10⁵, 4×10⁵, 3×10⁵, 2×10⁵, or 1×10⁵ or less bulk non-purified digest cells per tissue culture well.

In some aspects, the methods can further comprise the use of IL-2 in the culture. It is understood and herein contemplated that the concentration of the IL-2 can be adjusted to maximize the expansion of TILs. For example, the IL-2 concentration used to culture TILs can be 500, 600, 700, 800, 900, 1000, 1500, 2000, 2500, 3000, 3500, 4000, 4500, 5000, 5500, 6000, 6500, 7000, 7500, 8000 IU/mL or more.

The culture process employed by the art understood methods takes 5-7 weeks to expand TILs from bulk non-purified tumor digests. This is a significant problem in the art as additional time to initiating adoptive transfer therapy of TILs represents an increased risk to the patient due to progression of malignancy while the cell product is being prepared. Moreover, the added time needed for culturing requires additional resources of the hospital in additional personnel to requirements to maintain the culture and costs for media and maintaining a cleanroom. The present method decreases the expansion time to less than 5 weeks resulting in decreased attrition patients from therapy secondary to disease progression. For example, culturing to obtain an expanded population of TILs can occur for any time between 1 day and 5 weeks (35 days), preferably between 21 days (3 weeks) and 5 weeks (35 days), more preferably between 4 weeks (28 days) and 5 weeks (35 days). For example, the culture time can be less than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 days. In some aspect, the pre-REP expansion is harvested when the desired expansion is reached, but not more than 4 weeks. Thus, disclosed herein are TIL expansion methods wherein the pre-REP culture is 1, 2, 3, 4, 5, 6, 7 (1 week), 8, 9, 10, 11, 12, 13, 14 (2 weeks), 15, 16, 17, 18, 19, 20, 21 (3 weeks), 22, 23, 24, 25, 26, 27, or 28 (4 weeks) days. Following the pre-REP, the pre-REP TIL can be frozen and used at a later time. Ultimately, the fresh or thawed pre-REP TIL are submitted to a rapid expansion protocol (REP) which can last less than 1, 2, 3, 4, 5, 6, 7, 8, 9, 10, 11, 12, 13, 14 days. Where frozen TILs are used, the TILs can be thawed for 1-3 days. In some aspect, where thawed TILs are used, and the recovery of the thawed TILS is below 40×10⁶, a second culture of thawed TILs can be used to augment the number of TILs.

To maintain the quality of the nutrients in culture and remove any waste, it is understood and herein contemplated that the all or a portion of the media in the reservoir may be exchanged. The exchange of media can comprise 5, 10, 15, 20, 25, 30, 35, 40, 45, 50, 55, 60, 65, 70, 75, 80, 85, 90, 95, or 100% removal and replacement of media. This media exchange can be accomplished employing any acceptable method for proper tissue culture maintenance known in the art. In one aspect, the media exchange can occur at least one time during the culture of the TILs. For example, the media in the reservoir can be exchanged 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 times during the culture period. That is, the media exchange can occur once during the culture period, once every 15 days, once every 10 days, once every 7 days, once every 5 days, once every other day, or about 2 to 3 times per week.

The culture methods employed herein can utilize any complete media comprising IL-2 appropriate for the growth and propagation of the TILs, including, but not limited to Minimum Essential Medium (MEM), Eagles's Minimum Essential Medium (EMEM), Dulbecco's Minimum Essential Medium (DMEM) Medium 199, RPMI 1640, CMRL-1066, BGJb Medium, Iscove's Modified Dulbecco's Medium (IMDM), and Blood Cell Media.

The TILs can be cultured in any gas permeable reservoir suitable for cell culture and the expansion of TILs. In one aspect, it is understood and herein contemplated that large tissue culture flasks can slow down the expansion of TILs as it takes longer for cells to reach confluency. In one aspect, the gas permeable reservoir can be a tissue culture plate comprising 6 (approximately 10 cm² surface area per well and 60 cm² total surface area), 12(approximately 4 cm² surface area per well and approximately 48 cm² total surface area), 24 (approximately 2 cm² surface area per well and approximately 48 cm² total surface area), 48(approximately 1 cm² surface area per well and approximately 48 cm² total surface area), or 96 (approximately 0.32 cm² surface area per well and 31 cm² total surface area) wells (for example, G-Rex24 well plate or G-Rex6 well plate manufactured by Wilson Wolf). In some aspect, the plates can be silicone coated.

Also disclosed herein are methods of treating, inhibiting, reducing, decreasing, ameliorating, and/or preventing a cancer and/or metastasis, further adding autologous B cells to the culture of TILs and CD40L.

Also disclosed herein are methods of treating, inhibiting, reducing, decreasing, ameliorating, and/or preventing a cancer and/or metastasis, further comprising measuring the tumor gene expression level of chemokine (C-C motif) ligand 2 (CCL2), CCL3, CCL4, CCL5, CCL8, chemokine (C-C motif) ligand 18 (pulmonary and activation-regulated) (CCL18), CCL19, CCL21, chemokine (C-X-C motif) ligand 9 (CXCL9), CXCL10, CXCL11, and CXCL13 in tumor cells and comparing the tumor gene expression levels to reference gene expression levels; and identifying a subject who has tumor gene expression levels above the reference gene expression levels.

In one aspect, also disclosed herein are methods of treating, inhibiting, reducing, decreasing, ameliorating, and/or preventing a cancer and/or metastasis, further comprising adding autologous B cells to the culture of TILs and the one or more CD40 agonists.

Disclosed herein are methods of treating tumors in a subject. Tumors include malignancies of the various organ systems, such as affecting lung, breast, thyroid, lymphoid, gastrointestinal, and genito-urinary tract, as well as adenocarcinomas which include malignancies such as most colon cancers, renal-cell carcinoma, prostate cancer and/or testicular tumors, non-small cell carcinoma of the lung, cancer of the small intestine and cancer of the esophagus. In some embodiments, the cancer is a melanoma, breast, lung, colorectal, urothelial, or genitourinary cancer. The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. In some embodiments, the disease is renal carcinoma or melanoma. Exemplary carcinomas include those forming from tissue of the cervix, lung, prostate, breast, head and neck, colon and ovary. The term also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures. The term “sarcoma” is art recognized and refers to malignant tumors of mesenchymal derivation. In some embodiments of the methods described herein, the 5 tumor is a solid tumor.

The method comprises treating the patient with anti-CD40 antibodies or antigenbinding fragments thereof. The monoclonal antibodies have a strong affinity for CD40 and are characterized by a dissociation constant (Kd) of at least 10⁻⁵ M, preferably at least about 10⁻⁸ M to about 10⁻²⁰ M, more preferably at least about 5×10⁻⁹ to about 10⁻¹⁶ M. Suitable monoclonal antibodies have human constant regions; preferably they also have wholly or partially humanized framework regions; and most preferably are fully human antibodies or antigen-binding fragments thereof. Examples of such monoclonal antibodies are the antibody designated herein as 15B8, the monoclonal antibody produced by the hybridoma cell line designated 15B8, a monoclonal antibody comprising an amino acid sequence selected from the group consisting of DIVMTQSPLSLSVAPGQPASISCKSSQSLLESYGETYLYWYLQKPGQPPQLLIYAVFK RFSGVPDRFSGSGSGTDFTLKISRVEAEDVGVYYCMQSMQLPLTFGGGTKVEIK (SEQ ID NO:2) and QVQLQESGGGVVQPGRSLRLSCAASGFTFNNFGIHWVRQAPGKGLEWVAVISYDGS DKYYADSVKGRFTISRDNSKNTLNLQMNSLRAEDTAVYYCARDRRYYYHYYGMD VWGQGTMVTVSS (SEQ ID NO:4); a monoclonal antibody comprising an amino acid sequence encoded by a nucleic acid molecule comprising a nucleotide sequence selected from the group consisting of GATATTGTGATGACCCAGTCTCCACTCTCTCTGTCCGTCGCCCCTGGACAGCCGG CCTCCATCTCCTGTAAGTCTAGTCAGAGCCTCCTGGAGAGTTATGGAGAGACCTA TTTGTATTGGTACCTGCAGAAGCCAGGCCAGCCTCCACAGCTCCTGATCTATGCA GTTTTTAAGCGGTTCTCTGGAGTGCCAGATAGGTTCAGTGGCAGCGGGTCAGGGA CAGATTTCACACTGAAAATCAGCCGGGTGGAGGCTGAGGATGTTGGGGTTTATTA CTGCATGCAAAGTATGCAGCTTCCTCTCACTTTCGGCGGAGGGACCAAGGTGGA GATCAAA (SEQ ID NO:1) and CAGGTGCAGCTGCAGGAGTCTGGGGGAGGCGTGGTCCAGCCTGGGAGGTCCCTG AGACTCTCCTGTGCAGCCTCTGGATTCACCTTCAATAACTTTGGCATACACTGGG TCCGCCAGGCTCCAGGCAAGGGGCTGGAGTGGGTGGCAGTTATATCATATGATG GAAGTGATAAATATTATGCAGACTCCGTGAAGGGCCGATTCACCATCTCCAGAG ACAATTCCAAGAACACGCTGAATCTGCAAATGAATAGTCTGAGAGCTGAGGACA CGGCTGTGTATTACTGTGCGAGAGATCGTCGGTATTACTACCACTACTACGGTAT GGACGTCTGGGGCCAAGGGACCATGGTCACCGTCTCCTCA (SEQ ID NO:3); and antigen-binding fragments of these monoclonal antibodies that retain the capability of specifically binding to human CD40.

The disclosed anti-CD40 antibodies may be administered either alone, or as a pharmaceutical composition in combination with diluents and/or with other components such as IL-2, IL-15, or other cytokines or cell populations. Briefly, pharmaceutical compositions may comprise a target cell population as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions for use in the disclosed methods are in some embodiments formulated for intravenous administration. Pharmaceutical compositions may be administered in any manner appropriate treat the cancer. The quantity and frequency of administration will be determined by such factors as the condition of the patient, and the severity of the patient's disease, although appropriate dosages may be determined by clinical trials.

When “an immunologically effective amount”, “an anti-tumor effective amount”, “an tumor-inhibiting effective amount”, or “therapeutic amount” is indicated, the precise amount of the compositions of the present invention to be administered can be determined by a physician with consideration of individual differences in age, weight, tumor size, extent of infection or metastasis, and condition of the patient (subject). The cells can be administered by using infusion techniques that are commonly known in immunotherapy (see, e.g., Rosenberg et al., New Eng. J. of Med. 319:1676, 1988). The optimal dosage and treatment regime for a particular patient can readily be determined by one skilled in the art of medicine by monitoring the patient for signs of disease and adjusting the treatment accordingly.

In some embodiments, the therapy comprises administering to a patient a therapeutically effective dose of a pharmaceutical composition comprising suitable anti-CD40 antibodies or antigen-binding fragments thereof. A therapeutically effective dose of the anti-CD40 antibody or fragment thereof is in the range from about 0.01 mg/kg to about 40 mg/kg, from about 0.01 mg/kg to about 30 mg/kg, from about 0.1 mg/kg to about 30 mg/kg, from about 1 mg/kg to about 30 mg/kg, from about 3 mg/kg to about 30 mg/kg, from about 3 mg/kg to about 25 mg/kg, from about 3 mg/kg to about 20 mg/kg, from about 5 mg/kg to about 15 mg/kg, or from about 7 mg/kg to about 12 mg/kg. It is recognized that the treatment may comprise administration of a single therapeutically effective dose or administration of multiple therapeutically effective doses of the anti-CD40 antibody or antigen-binding fragment thereof.

The administration of the disclosed compositions may be carried out in any convenient manner, including by injection, transfusion, or implantation. The compositions described herein may be administered to a patient subcutaneously, intradermally, intratumorally, intranodally, intramedullary, intramuscularly, by intravenous (i.v.) injection, or intraperitoneally. In some embodiments, the disclosed compositions are administered to a patient by intradermal or subcutaneous injection. In some embodiments, the disclosed compositions are administered by i.v. injection. The compositions may also be injected directly into a tumor, lymph node, or site of infection.

In certain embodiments, the disclosed anti-CD40 antibodies are administered to a patient in conjunction with chemotherapy, therapeutic tumour vaccines, agitation of Toll-like receptors, cytokine therapy, and/or blockades of immune checkpoint inhibitors.

In certain embodiments, the disclosed anti-CD40 antibodies are administered to a patient in conjunction with carboplatin, cisplatin, etoposide, gemcitabine, ifosfamide, paclitaxel, and/or pemetrexed.

In certain embodiments, the disclosed anti-CD40 antibodies are administered to a patient in conjunction with atezolizumab, cabiralizumab, emactuzumab, nivolumab, pembrolizumab, rituximab, tremelimumab, and/or vanucizumab. In certain embodiments, the disclosed anti-CD40 antibodies are administered to a patient in conjunction with (e.g., before, simultaneously or following) any number of relevant treatment modalities, including but not limited to thalidomide, dexamethasone, bortezomib, and lenalidomide. In further embodiments, the anti-CD40 antibodies may be used in combination with chemotherapy, radiation, immunosuppressive agents, such as cyclosporin, azathioprine, methotrexate, mycophenolate, and FK506, antibodies, or other immunoablative agents such as CAM PATH, anti-CD3 antibodies or other antibody therapies, cytoxin, fludaribine, cyclosporin, FK506, rapamycin, mycophenolic acid, steroids, FR901228, cytokines, and irradiation. In some embodiments, the TILs are administered to a patient in conjunction with (e.g., before, simultaneously or following) bone marrow transplantation, T cell ablative therapy using either chemotherapy agents such as, fludarabine, external-beam radiation therapy (XRT), cyclophosphamide, or antibodies such as OKT3 or CAMPATH. In another embodiment, the cell compositions of the present invention are administered following B-cell ablative therapy such as agents that react with CD20, e.g., Rituxan.

The cancer of the disclosed methods can be any cell in a subject undergoing unregulated growth, invasion, or metastasis. Cancers include prostate cancer, ovarian cancer, adenocarcinoma of the lung, breast cancer, endometrial cancer, gastric cancer, colon cancer, and pancreatic cancer. In some cases, the cancer comprises myelodysplastic syndrome, acute myeloid leukemia, or bi-phenotypic leukemia.

In some aspects, the cancer can be any neoplasm or tumor for which radiotherapy is currently used. Alternatively, the cancer can be a neoplasm or tumor that is not sufficiently sensitive to radiotherapy using standard methods. Thus, the cancer can be a sarcoma, lymphoma, leukemia, carcinoma, blastoma, or germ cell tumor. A representative but non-limiting list of cancers that the disclosed compositions can be used to treat include lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, kidney cancer, lung cancers such as small cell lung cancer and non-small cell lung cancer, neuroblastoma/glioblastoma, ovarian cancer, pancreatic cancer, prostate cancer, skin cancer, liver cancer, melanoma, squamous cell carcinomas of the mouth, throat, larynx, and lung, endometrial cancer, cervical cancer, cervical carcinoma, breast cancer, epithelial cancer, renal cancer, genitourinary cancer, pulmonary cancer, esophageal carcinoma, head and neck carcinoma, large bowel cancer, hematopoietic cancers; testicular cancer; colon and rectal cancers, prostatic cancer, and pancreatic cancer.

The disclosed anti-CD40 antibodies can be used in combination with any compound, moiety or group which has a cytotoxic or cytostatic effect. Drug moieties include chemotherapeutic agents, which may function as microtubulin inhibitors, mitosis inhibitors, topoisomerase inhibitors, or DNA intercalators, and particularly those which are used for cancer therapy.

The disclosed anti-CD40 antibodies can be used in combination with a checkpoint inhibitor. The two known inhibitory checkpoint pathways involve signaling through the cytotoxic T-lymphocyte antigen-4 (CTLA-4) and programmed-death 1 (PD-1) receptors. These proteins are members of the CD28-B7 family of cosignaling molecules that play important roles throughout all stages of T cell function. The PD-1 receptor (also known as CD279) is expressed on the surface of activated T cells. Its ligands, PD-L1 (B7-H1; CD274) and PD-L2 (B7-DC; CD273), are expressed on the surface of APCs such as dendritic cells or macrophages. PD-L1 is the predominant ligand, while PD-L2 has a much more restricted expression pattern. When the ligands bind to PD-1, an inhibitory signal is transmitted into the T cell, which reduces cytokine production and suppresses T-cell proliferation. Checkpoint inhibitors include, but are not limited to antibodies that block PD-1 (Nivolumab (BMS-936558 or MDX1106), CT-011, MK-3475), PD-L1 (MDX-1105 (BMS-936559), MPDL3280A, MSB0010718C), PD-L2 (rHIgM12B7), CTLA-4 (Ipilimumab (MDX-010), Tremelimumab (CP-675,206)), IDO, B7-H3 (MGA271),

B7-H4, TIM3, LAG-3 (BMS-986016).

Human monoclonal antibodies to programmed death 1 (PD-1) and methods for treating cancer using anti-PD-1 antibodies alone or in combination with other immunotherapeutics are described in U.S. Pat. No. 8,008,449, which is incorporated by reference for these antibodies. Anti-PD-L1 antibodies and uses therefor are described in U.S. Pat. No. 8,552,154, which is incorporated by reference for these antibodies. Anticancer agent comprising anti-PD-1 antibody or anti-PD-L1 antibody are described in U.S. Pat. No. 8,617,546, which is incorporated by reference for these antibodies.

In some embodiments, the PDL1 inhibitor comprises an antibody that specifically binds PDL1, such as BMS-936559 (Bristol-Myers Squibb) or MPDL3280A (Roche). In some embodiments, the PD1 inhibitor comprises an antibody that specifically binds PD1, such as lambrolizumab (Merck), nivolumab (Bristol-Myers Squibb), or MEDI4736 (AstraZeneca). Human monoclonal antibodies to PD-1 and methods for treating cancer using anti-PD-1 antibodies alone or in combination with other immunotherapeutics are described in U.S. Pat. No. 8,008,449, which is incorporated by reference for these antibodies. Anti-PD-L1 antibodies and uses therefor are described in U.S. Pat. No. 8,552,154, which is incorporated by reference for these antibodies. Anticancer agent comprising anti-PD-1 antibody or anti-PD-L1 antibody are described in U.S. Pat. No. 8,617,546, which is incorporated by reference for these antibodies.

The disclosed anti-CD40 antibodies can be used in combination with other cancer immunotherapies. There are two distinct types of immunotherapy: passive immunotherapy uses components of the immune system to direct targeted cytotoxic activity against cancer cells, without necessarily initiating an immune response in the patient, while active immunotherapy actively triggers an endogenous immune response. Passive strategies include the use of the monoclonal antibodies (mAbs) produced by B cells in response to a specific antigen. The development of hybridoma technology in the 1970s and the identification of tumor-specific antigens permitted the pharmaceutical development of mAbs that could specifically target tumor cells for destruction by the immune system. Thus far, mAbs have been the biggest success story for immunotherapy; the top three best-selling anticancer drugs in 2012 were mAbs. Among them is rituximab (Rituxan, Genentech), which binds to the CD20 protein that is highly expressed on the surface of B cell malignancies such as non-Hodgkin's lymphoma (NHL). Rituximab is approved by the FDA for the treatment of NHL and chronic lymphocytic leukemia (CLL) in combination with chemotherapy. Another important mAb is trastuzumab (Herceptin; Genentech), which revolutionized the treatment of HER2 (human epidermal growth factor receptor 2)-positive breast cancer by targeting the expression of HER2.

Generating optimal “killer” CD8 TIL responses may also require T cell receptor activation plus co-stimulation, which can be provided through ligation of tumor necrosis factor receptor family members, including OX40 (CD134) and 4-1BB (CD137). OX40 is of particular interest as treatment with an activating (agonist) anti-OX40 mAb augments T cell differentiation and cytolytic function leading to enhanced anti-tumor immunity against a variety of tumors.

In some embodiments, such an additional therapeutic agent may be selected from an antimetabolite, such as methotrexate, 6-mercaptopurine, 6-thioguanine, cytarabine, fludarabine, 5-fluorouracil, decarbazine, hydroxyurea, asparaginase, gemcitabine or cladribine.

In some embodiments, such an additional therapeutic agent may be selected from an alkylating agent, such as mechlorethamine, thioepa, chlorambucil, melphalan, carmustine (BSNU), lomustine (CCNU), cyclophosphamide, busulfan, dibromomannitol, streptozotocin, dacarbazine (DTIC), procarbazine, mitomycin C, cisplatin and other platinum derivatives, such as carboplatin.

In some embodiments, such an additional therapeutic agent may be selected from an anti-mitotic agent, such as taxanes, for instance docetaxel, and paclitaxel, and vinca alkaloids, for instance vindesine, vincristine, vinblastine, and vinorelbine.

In some embodiments, such an additional therapeutic agent may be selected from a topoisomerase inhibitor, such as topotecan or irinotecan, or a cytostatic drug, such as etoposide and teniposide.

In some embodiments, such an additional therapeutic agent may be selected from a growth factor inhibitor, such as an inhibitor of ErbB1 (EGFR) (such as an EGFR antibody, e.g. zalutumumab, cetuximab, panitumumab or nimotuzumab or other EGFR inhibitors, such as gefitinib or erlotinib), another inhibitor of ErbB2 (HER2/neu) (such as a HER2 antibody, e.g. trastuzumab, trastuzumab-DM 1 or pertuzumab) or an inhibitor of both EGFR and HER2, such as lapatinib).

In some embodiments, such an additional therapeutic agent may be selected from a tyrosine kinase inhibitor, such as imatinib (Glivec, Gleevec STI571) or lapatinib.

Therefore, in some embodiments, a disclosed antibody is used in combination with ofatumumab, zanolimumab, daratumumab, ranibizumab, nimotuzumab, panitumumab, hu806, daclizumab (Zenapax), basiliximab (Simulect), infliximab (Remicade), adalimumab (Humira), natalizumab (Tysabri), omalizumab (Xolair), efalizumab (Raptiva), and/or rituximab.

In some embodiments, a therapeutic agent for use in combination with TILs for treating the disorders as described above may be an anti-cancer cytokine, chemokine, or combination thereof. Examples of suitable cytokines and growth factors include IFNy, IL-2, IL-4, IL-6, IL-7, IL-10, IL-12, IL-13, IL-15, IL-18, IL-23, IL-24, IL-27, IL-28a, IL-28b, IL-29, KGF, IFNa (e.g., INFa2b), IFN, GM-CSF, CD40L, Flt3 ligand, stem cell factor, ancestim, and TNFa. Suitable chemokines may include Glu-Leu-Arg (ELR)-negative chemokines such as IP-10, MCP-3, MIG, and SDF-la from the human CXC and C-C chemokine families. Suitable cytokines include cytokine derivatives, cytokine variants, cytokine fragments, and cytokine fusion proteins.

In some embodiments, a therapeutic agent for use in combination with anti-CD40 antibodies for treating cancers as described above may be a cell cycle control/apoptosis regulator (or “regulating agent”). A cell cycle control/apoptosis regulator may include molecules that target and modulate cell cycle control/apoptosis regulators such as (i) cdc-25 (such as NSC 663284), (ii) cyclin-dependent kinases that overstimulate the cell cycle (such as flavopiridol (L868275, HMR1275), 7-hydroxystaurosporine (UCN-01, KW-2401), and roscovitine (R-roscovitine, CYC202)), and (iii) telomerase modulators (such as BIBR1532, SOT-095, GRN163 and compositions described in for instance U.S. Pat. Nos. 6,440,735 and 6,713,055). Non-limiting examples of molecules that interfere with apoptotic pathways include TNF-related apoptosis-inducing ligand (TRAIL)/apoptosis-2 ligand (Apo-2L), antibodies that activate TRAIL receptors, IFNs, and anti-sense Bcl-2.

In some embodiments, a therapeutic agent for use in combination with anti-CD40 antibodies for treating cancers as described above may be a hormonal regulating agent, such as agents useful for anti-androgen and anti-estrogen therapy. Examples of such hormonal regulating agents are tamoxifen, idoxifene, fulvestrant, droloxifene, toremifene, raloxifene, diethylstilbestrol, ethinyl estradiol/estinyl, an antiandrogene (such as flutaminde/eulexin), a progestin (such as such as hydroxyprogesterone caproate, medroxy-progesterone/provera, megestrol acepate/megace), an adrenocorticosteroid (such as hydrocortisone, prednisone), luteinizing hormone-releasing hormone (and analogs thereof and other LHRH agonists such as buserelin and goserelin), an aromatase inhibitor (such as anastrazole/arimidex, aminoglutethimide/cytraden, exemestane) or a hormone inhibitor (such as octreotide/sandostatin).

In some embodiments, a therapeutic agent for use in combination with anti-CD40 antibodies for treating the cancers as described above may be an anti-cancer nucleic acid or an anti-cancer inhibitory RNA molecule.

Combined administration, as described above, may be simultaneous, separate, or sequential. For simultaneous administration the agents may be administered as one composition or as separate compositions, as appropriate.

In some embodiments, the disclosed anti-CD40 antibodies are administered in combination with radiotherapy. Radiotherapy may comprise radiation or associated administration of radiopharmaceuticals to a patient is provided. The source of radiation may be either external or internal to the patient being treated (radiation treatment may, for example, be in the form of external beam radiation therapy (EBRT) or brachytherapy (BT)). Radioactive elements that may be used in practicing such methods include, e.g., radium, cesium-137, iridium-192, americium-241, gold-198, cobalt-57, copper-67, technetium-99, iodide-123, iodide-131, and indium-111.

In some embodiments, the disclosed anti-CD40 antibodies are administered in combination with surgery.

### G. EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

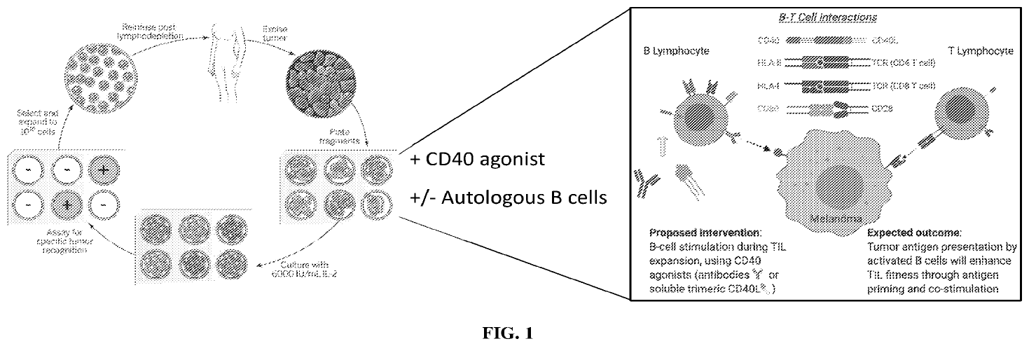

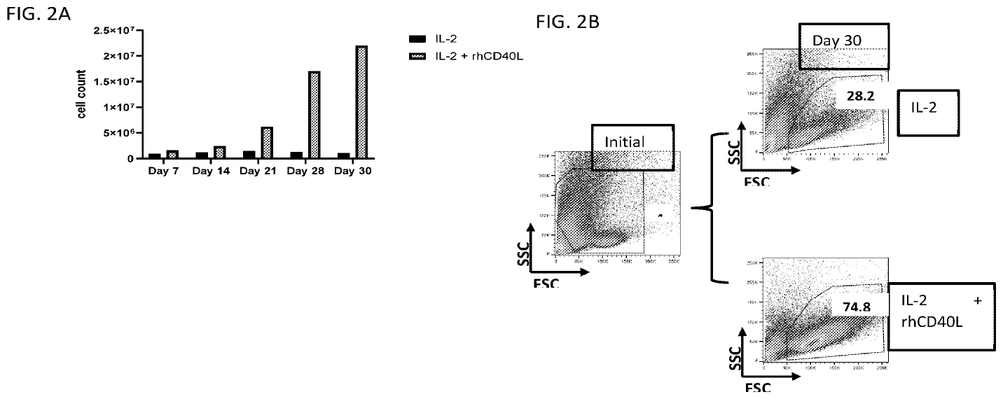

FIG. 1 shows TIL enhancement via ex vivo stimulation with CD40 agonists. Our observations from this process are that B-cells are frequently found in melanoma metastases, and they display signs of antigen experience and interaction with T-cells. Melanoma infiltrating B-cells express co-stimulatory ligands upon CD40 stimulation, and can present antigen to T cells. Importantly, they do not produce IL-10. Additionally, Frozen tumor digests grown in presence of CD40 agonists plus IL-2 expand significantly better than with IL-2 alone. From this we conclude that stimulation of B-cells and/or myeloid cells using CD40 agonists enhances the number and quality of ex vivo expanded TIL. Thus, we developed a TIL manufacturing process that includes CD40 stimulation during TIL expansion. In some instances, we add autologous B cells derived from peripheral blood, in addition to CD40 stimulation. To test the effect of CD40 stimulation on TIL expansion we added recombinant human CD40L to a culture of TILs and measured the effect on expansion (see FIG. 2). The results showed culture of TILs with IL-2 alone produced a maximum number of TILS at day 21 and never resulted in more than 2×10⁶ TILs. By contrast, the cultures including the addition of CD40L resulted in 5×10⁶ TILs at day 21 and kept expanding to over 2×10⁷ TILs by day 30 when we concluded the study.

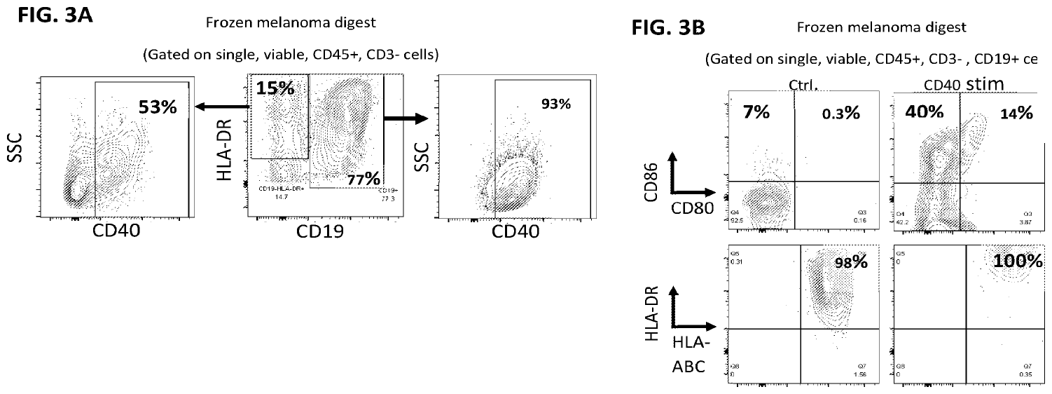

We hypothesized that the expansion of TILs in the presence of CD40L was in part through the effect of CD40L on B cells in the biopsy. As shown in FIG. 3, melanoma tumors contain B cells, which can be activated using a CD40 agonist. FIG. 3A shows frozen tumor digests were analyzed for the presence of B cells (CD19+) and myeloid cells (HLA-DR^{hi}). The vast majority of B cells expressed CD40, while half of myeloid cells did. FIG. 3B shows tumor digests were cultured for two days in presence of standard TIL expansion media (Ctrl.) or the same media supplemented with a CD40 agonist (CD40 stim). Flow cytometry analysis showed induction of CD80 and CD86 costimulatory ligands by the CD40 agonist. HLA molecules were detected in all B cells in resting and stimulated conditions, but the intensity of HLA-DR expression was increased in the CD40-stimulated samples.

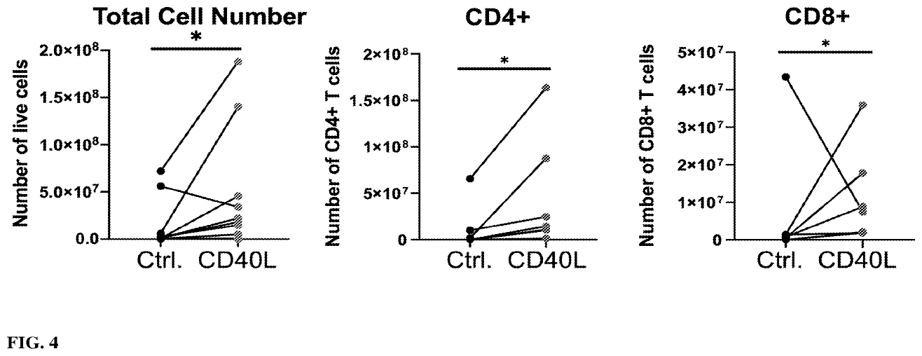

We next looked at the expansion of TILs for a melanoma digest in the presence of CD40 stimulation (FIG. 4). Frozen tumor digests were cultured in presence of high-dose IL-2 (control) or high-dose IL-2 plus a CD40 agonist for 4 weeks. We observed that the total number of live cells, CD4+ T lymphocytes, and of CD8+ lymphocytes was significantly increased in the CD40 stimulated group. *p<0.05.

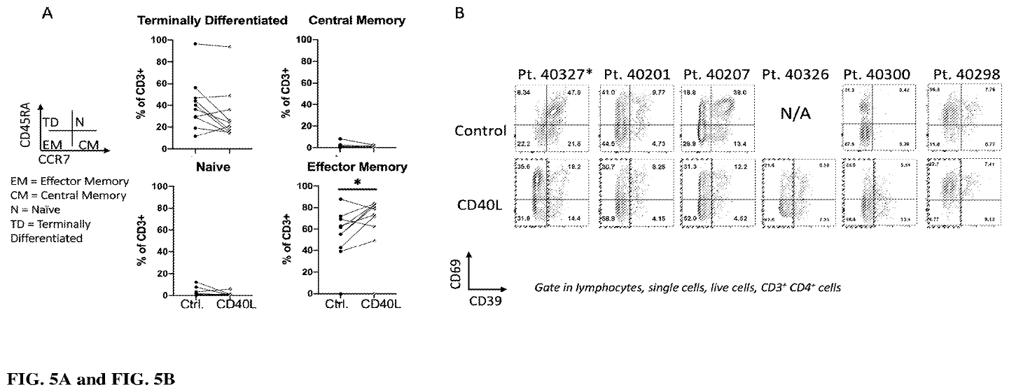

Next, we wanted to characterize the expanded TIL population to determine if the CD40 stimulation effected any population of T cells more than others. We found that CD40 stimulation results in increased Effector Memory cells, and increased CD39-cells in 3/5 cases (FIG. 5). Frozen tumor digests were cultured in presence of high-dose IL-2 (control) or high-dose IL-2 plus a CD40 agonist for 4 weeks. FIG. 5A shows analysis of differentiation phenotype based on CD45RA and CCR7 expression. FIG. 5B shows expression of CD39 and CD69 in the CD4 T cell compartment. *p<0.05.

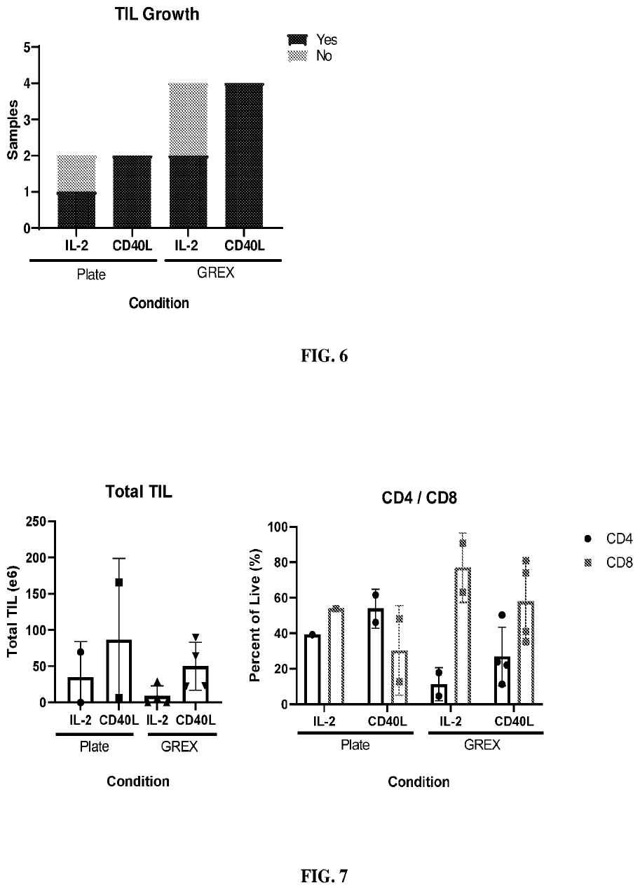

To see if CD40L could recue TIL growth in a culture of TILs stimulated in Il-2 alone and to observe the effect of permeable membrane in the culture well, we grew TILs with and without CD40L and on plates or G-REX (gas permeable) plates. As shown in FIG. 6 TILs cultured in the presence of CD40L showed a two-fold increase in expansion relative to IL-2 alone. Additionally the use of a gas permeable plate such as a G-REX plate increased TIL expansion two-fold more (i.e., 4-fold total). As shown in FIG. 7 the expansion of total TILs was significant in the presence of CD40L and 11-2 relative to IL-2 alone, but the increase was larger in CD4 T cells slightly changing the relative proportions of CD4 T cells to CD8 T cells. This change appeared to hold whether using plates or gas-permeable plates (e.g., G-REX).

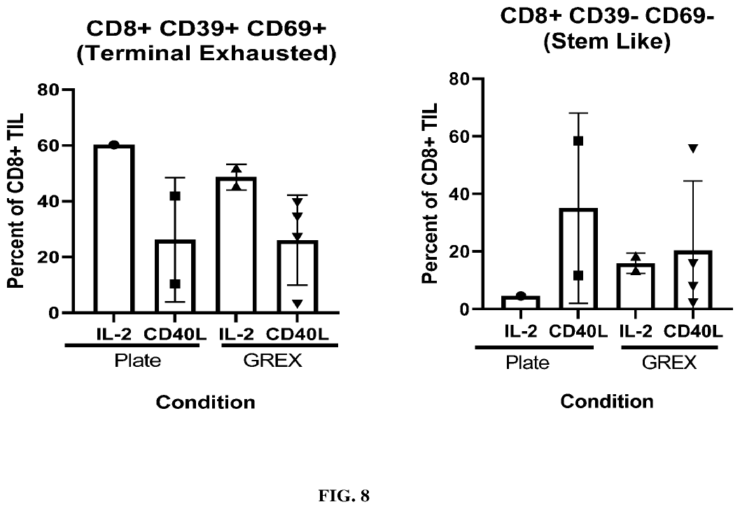

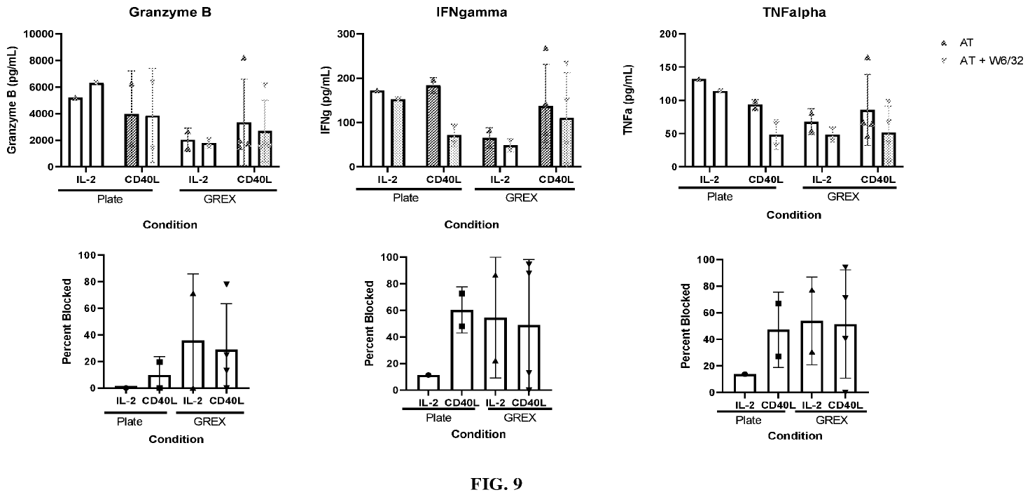

Further characterizing the CD8⁺ T cells in the TIL population, we observed that the percentage of stem-like CD8 T cells (CD39-CD69-CD8+ T cells) increased in the presence of CD40L, while the percentage of terminally exhausted CD8+ T cells (CD39+CD69+) decreased in the presence of CD40L (FIG. 8). We also looked at effect of CD40 stimulation using CD40L on effector cytokine secretion. TILs from NSCLC samples expanded in IL-2 or IL-2+CD40L were co-cultured with dissociated autologous tumor cells at a ratio of 1:1. An MHC-I blocking antibody was used for analysis of MHC-I specificity of reaction. After 24 hrs post culture, supernatant was collected. Using the ELLA platform, we measured Granzyme B, IFNγ, and TNFα. We did not observe a significant effect on cytokine secretion.

1. A method of expanding tumor infiltrating lymphocytes (TILs) in vitro or ex vivo comprising obtaining TILs and culturing the TILs in media comprising one or more CD40 agonists.

2. The method of expanding TILs of claim 1, wherein the CD40 agonist comprises CD40 ligand (CD40L).

3. The method of expanding TILs of claim 1, wherein the TILs are cultured for at least 21 days.

4. The method of expanding TILs of claim 1, further adding autologous B cells to the culture of TILs and CD40L.

5. The method of claim 1 wherein the TILs are cultured in a gas permeable reservoir.

6. A method of expanding tumor infiltrating lymphocytes (TILs) in in a subject with a tumor comprising administering one or more CD40 agonists to the subject at the site of the tumor.

7. The method of expanding tumor infiltrating lymphocytes (TILs) in in a subject with a tumor of claim 6, wherein the CD40 agonist comprises CD40 ligand (CD40L).

8. The method of claim 6 wherein the TILs are cultured in a gas permeable reservoir.

9. A method of treating a cancer in a subject comprising obtaining tumor infiltrating lymphocytes (TILs) from the subject; culturing the TILs in media comprising one or more CD40 agonists, and administering the cultured TILs to the subject.

10. The method of treating a cancer of claim 9, wherein the one or more CD40 agonist comprises CD40 ligand (CD40L).

11. The method of treating a cancer of claim 9, wherein the TILs are cultured for at least 21 days.

12. The method of treating a subject with a cancer of claim 9, further comprising measuring the tumor gene expression level of chemokine (C-C motif) ligand 2 (CCL2), CCL3, CCL4, CCL5, CCL8, chemokine (C-C motif) ligand 18 (pulmonary and activation-regulated) (CCL18), CCL19, CCL21, chemokine (C-X-C motif) ligand 9 (CXCL9), CXCL10, CXCL11, and CXCL13 in tumor cells and comparing the tumor gene expression levels to reference gene expression levels; and identifying a subject who has tumor gene expression levels above the reference gene expression levels.

13. The method of treating a cancer of claim 9, further comprising adding autologous B cells to the culture of TILs and the one or more CD40 agonists.

14. The method of claim 9 wherein the TILs are cultured in a gas permeable reservoir.
